(19) United States
(12) Patent Application Publication
(10) Pub. No.: US 2023/0242630 A1
(43) Pub. Date: Aug. 3, 2023

(54) MODULATING IMMUNE RESPONSE

(71) Applicant: THE COUNCIL OF THE QUEENSLAND INSTITUTE OR MEDICAL RESEARCH, Queensland (AU)

(72) Inventors: Susanna Shu Szen NG, Queensland (AU); Christian Robert ENGWERDA, Queensland (AU)

(21) Appl. No.: 17/055,457

(22) PCT Filed: Jan. 24, 2019
(86) PCT No.: PCT/AU2019/050049

§ 371 Date: Nov. 13, 2020

(30) Foreign Application Priority Data
May 15, 2018 (AU) 2018901677

(51) Int. Cl. C07K 16/18; A61P 35/00; A61K 39/395; A61K 31/713; A61K 39/00

(52) U.S. Cl. CPC C07K 16/18; A61P 35/00; A61K 39/39533; A61K 31/713; A61K 2039/505

(57) ABSTRACT

The present disclosure relates to methods and compositions for modulating immune responses, methods of treating or preventing diseases such as autoimmune and inflammatory disorders and cancer, as well as methods of enhancing immune responses to antigens and for the treatment of infectious diseases.

### TECHNICAL FIELD

The present disclosure relates to methods and compositions for modulating immune responses, methods of treating or preventing diseases such as autoimmune and inflammatory disorders and cancer, as well as methods of enhancing immune responses to antigens and for the treatment of infectious diseases.

### BACKGROUND

Inflammation is part of the complex biological response of vascular tissues to harmful stimuli, such as pathogens, damaged cells, or irritants. Inflammation is a protective attempt by the organism to remove the injurious stimuli and to initiate the healing process. Without inflammation, wounds and infections would never heal. Similarly, progressive destruction of the tissue would compromise the survival of the organism. However, chronic inflammation can also lead to a host of diseases, such as rheumatoid arthritis, atherosclerosis, and even cancer.

An autoimmune disorder is a condition that occurs when the immune system mistakenly attacks and destroys healthy body tissue. There are more than 80 different types of autoimmune disorders. In patients with an autoimmune disorder, the system reacts to normal body tissues that it would normally ignore.

Cancer immunotherapy encompasses a diverse variety of treatment approaches including passive administration of tumor-specific monoclonal antibodies and other immune system components, active immunization to elicit or augment specific T cell-mediated immune responses against tumor cells, adoptive transfer of ex vivo modified T cells, and non-specific enhancement of immune responsiveness with immune modulatory agents Immunotherapy has already had a major impact on the management of a broad range of cancers, however, the field of cancer immunotherapy is complex and rapidly evolving Immunotherapies differ from conventional chemotherapy in their mechanisms of action as well as the types of responses produced, and many active immunotherapies incorporate multiple components (e.g. antigens, adjuvants, and delivery vehicles).

It is increasingly recognized that robust therapeutic activity requires that immunization is combined with other immunomodulatory strategies directed at enhancing general immune responsiveness and overcoming the immunosuppressive mechanisms through which tumours promote a tolerogenic environment.

Thus, there remains a need for methods of modulating a patient's immune response in the treatment of autoimmune and inflammatory diseases and cancer, as well as during immunization.

### SUMMARY

The present inventors investigated the expression of Natural Killer Cell Granule Protein 7 (NKG7) in immune cells and have determined that the loss of NKG7 expression results in reduced blood concentrations of pro-inflammatory cytokines and an increase in anti-inflammatory cytokines.

Accordingly, in one aspect the present disclosure provides a method of modulating an immune response in a subject, the method comprising administering to the subject a compound that modulates NKG7 activity.

In some embodiments, the compound inhibits activity of NKG7.

In one embodiment, the compound is an antibody that binds NKG7 and inhibits NKG7 activity.

In some embodiments, the compound increases NKG7 activity.

In one embodiment, the compound is an antibody that binds NKG7 and increases NKG7 activity.

In some embodiments, the compound is an NKG7 polypeptide or fragment thereof.

In some embodiments, the antibody that modulates NKG7 activity binds extracellular loop 1 or extracellular loop 2 of NKG7. In one particular embodiment, the antibody binds an epitope comprising one or more amino acids in SEQ ID NO:11, 12, 13 or 14.

In another aspect, the present disclosure provides a method of modulating an immune response and/or treating or preventing an autoimmune disease or inflammatory condition in a subject by modulating expression of NKG7 in subject cells, the method comprising editing the NKG7 gene or other DNA sequences that encode NKG7 regulatory elements of the NKG7 gene, resulting in a modulation of expression or function of the NKG7 gene in the subject cells.

In one embodiment, the method is performed ex-vivo on cells obtained from the subject, wherein the editing comprises introducing into the cells one or more deoxyribonucleic acid (DNA) endonucleases to effect one or more single-strand breaks (SSBs) or double-strand breaks (DSBs) within or near the NKG7 gene or other DNA sequences that encode regulatory elements of the NKG7 gene that results in a permanent deletion, insertion, correction, or modulation of expression or function of one or more mutations or exons within or near or affecting the expression or function the NKG7 gene or other DNA sequences that encode regulatory elements of the NKG7 gene, and the method comprises returning the cells to the subject.

In another embodiment, the method is performed in vivo and the editing is performed by delivering a gene editing system in vivo to the subject.

In some embodiments, the gene editing system is the CRISPR/Cas9 gene editing system.

In another aspect, the present disclosure provides a method of treating or preventing an autoimmune disease or inflammatory condition in a subject, the method comprising administering to the subject a compound that inhibits NKG7 activity.

In some embodiments, the autoimmune disease or inflammatory condition is selected from graft versus host disease (GVHD), rheumatoid arthritis, inflammatory bowel disease (IBD), lupus, neuro-inflammation, multiple sclerosis, Alzheimer's disease, Parkinson's disease and systemic inflammatory response syndrome (SIRS).

In one embodiment, the autoimmune disease or inflammatory condition is rheumatoid arthritis.

In another embodiment, the autoimmune disease or inflammatory condition is an inflammatory bowel disease (IBD).

In one embodiment, the inflammatory bowel disease is Crohn's disease.

In another embodiment, the inflammatory bowel disease is ulcerative colitis.

In another aspect, the present disclosure provides a method of reducing the risk of graft-versus-host disease (GVHD) in a transplant recipient receiving an allogeneic transplant, the method comprising administering a compound that inhibits NKG7 activity to the transplant recipient.

In some embodiments, the compound that inhibits NKG7 activity is administered to the transplant recipient prior to receiving the allogeneic transplant.

In some embodiments, the compound that inhibits NKG7 activity is administered to the transplant recipient at the time of, or after receiving the allogeneic transplant.

In one embodiment, the compound is an antibody that binds NKG7 and inhibits NKG7 activity.

In another aspect, the present disclosure provides a method of treating cancer in a subject, the method comprising administering to the subject a compound that increases NKG7 activity in the subject.

In some embodiments, the compound is an antibody that binds NKG7 and increases NKG7 activity.

In some embodiments, the subject is treated with a cancer immunotherapy.

In some embodiments, the cancer immunotherapy is selected from immune checkpoint inhibitor therapy, and antibody therapy.

In one aspect, the present disclosure provides a method of enhancing an immune response to a vaccine antigen in a subject, the method comprising administering to the subject:

i) a compound that increases NKG7 activity, and

ii) a vaccine antigen.

In some embodiments, NKG7 comprises an amino acid sequence at least 90% identical to any one of SEQ ID NOs:1, 3, 5, 7, or 9 and which has NKG7 activity.

In some embodiments, NKG7 is encoded by a nucleic acid molecule which hybridises under stringent conditions with a nucleic acid comprising a sequence selected from any one of SEQ ID NOs:2, 4, 6, 8, or 10.

In some embodiments, NKG7 consists of a sequence selected from any one of SEQ ID NOs:1, 3, 5, 7, or 9 and having substitution of, or deletion or addition of, one or several amino acids.

In one aspect, the present disclosure provides a pharmaceutical composition for treating or preventing an autoimmune disease or inflammatory condition, the pharmaceutical composition comprising a compound that inhibits NKG7 activity and a pharmaceutically acceptable carrier.

In another aspect, the present disclosure provides a pharmaceutical composition for treating cancer, the pharmaceutical composition comprising a compound that increases NKG7 activity and a pharmaceutically acceptable carrier.

In another aspect, the present disclosure provides use of a compound that inhibits activity of NKG7 in the manufacture of a medicament for the treatment of an autoimmune disease or inflammatory condition.

In another aspect, the present disclosure provides use of a compound that increases activity of NKG7 in the manufacture of a medicament for the treatment of cancer.

In another aspect, the present disclosure provides an immunogenic composition comprising a compound that increases NKG7 activity.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### KEY TO THE SEQUENCE LISTING

SEQ ID NO: 1—Amino acid sequence of NKG7 polypeptide
SEQ ID NO:2—NKG7 nucleic acid coding sequence
SEQ ID NO:3—Amino acid sequence of NKG7 polypeptide isoform 1
SEQ ID NO:4—Nucleic acid coding sequence of NKG7 isoform 1
SEQ ID NO:5—Amino acid sequence of NKG7 polypeptide variant
SEQ ID NO:6—Nucleic acid coding sequence of NKG7 polypeptide variant
SEQ ID NO:7—Amino acid sequence of murine NKG7 polypeptide
SEQ ID NO:8—Nucleic acid coding sequence of murine NKG7 polypeptide
SEQ ID NO:9—Amino acid sequence of murine NKG7 polypeptide variant
SEQ ID NO:10—Nucleic acid coding sequence of murine NKG7 polypeptide variant
SEQ ID NO:11—Predicted amino acid sequence of extracellular loop 1
SEQ ID NO:12—Predicted amino acid sequence of extracellular loop 2
SEQ ID NO:13—Predicted amino acid sequence of murine extracellular loop 1
SEQ ID NO:14—Predicted amino acid sequence of murine extracellular loop 2

### BRIEF DESCRIPTION OF THE DRAWINGS

FIG. 1. CD4⁺ T cells from the liver (site of acute, resolving infection) and spleen (site of chronic infection) of naïve and Leishmania donovani-infected C57BL/6 mice, and from the peripheral blood mononuclear cells (PBMCs) of visceral leishmaniasis (VL) patients during infection and post-treatment. Mouse cells were isolated by fluorescence-activated cell sorting (FACS) and patient cells were isolated by magnetic activated cell sorting (MACS) (A-C). Comparison of infected mouse and human data sets with either corresponding naïve cell populations (mouse) or corresponding cell populations 30 days after drug treatment (patients) identified 1816, 2748, and 5784 differentially expressed genes in CD4⁺ T cells from the spleen, liver, and PBMCs respectively (D and E).

FIG. 2. The waterfall plot shows the upregulated genes in the chronic signature (differentially expressed genes common between CD4⁺ T cells isolated from mouse spleen and human peripheral blood mononuclear cells (PBMCs) (A). NKG7 was the most upregulated gene in CD4⁺ T cells isolated from mouse spleen. The cellular map displays cellular localisation information based on Gene Ontology: Cellular Compartment information (B). NKG7 is known to encode a membrane protein. The increase in NKG7 expression was validated by real-time qPCR in CD4⁺ T cells isolated from the PBMCs of patients with Visceral Leishmaniasis (VL) and endemic controls (EC), as well as Foxp3⁻ CD4⁺ T conventional (Tconv) cells and Foxp3⁺ CD4⁺ T regulatory cells (Tregs) from the spleen and liver of L. donovani-infected mice (C).

FIG. 3. Data from The Cancer Genome Atlas (TCGA) shows the differential expression of NKG7 within the tumour from various cancers, most notably kidney renal clear cell carcinoma (KIRC) and Glioblastoma (GBM) (A; adapted from http://firebrowse.org/). While expression data is obtained from tumour biopsies, there is little evidence of mutation sites on NKG7 that confer cancer risk (B; adapted from http://www.cbioportal.org/output). Stratification of patients from a skin cutaneous melanoma cohort (TGCA: SKCM) into high NKG7 expressors (highest quartile) and low NKG7 expressors (lowest quartile) reveals a significant increase in survival probability in the group with high NKG7 expression (C; shaded area indicates confidence interval).

FIG. 4. The mouse model B6.Nkg7-cre was generated (A), and was crossed to the B6.membrane tdTomato/membrane GFP (mT/mG) to produce the reporter mouse Nkg7-cre×mT/mG (B). The expression of Nkg7 in Cre-expressing (+) mice, determined by the surrogate marker GFP, is shown on natural killer cells in the naïve state.

FIG. 5. B6N.Nkg7-KO mice were infected with L. donovani. At day 14 post-infection (p.i.), the effects of Nkg7 deficiency were quantified. Mice that were deficient in Nkg7 exhibited lower spleen and liver weights compared to C57BL/6N (wild-type) mice (A). Parasite burden in Nkg7-deficient mice were markedly elevated compared to wild-type mice (B). The levels of the inflammation-promoting cytokines interferon (IFN)γ, tumour necrosis factor (TNF), and IL-6 were also found to be lower in the serum of Nkg7-deficient mice compared to wild-type mice (C).

FIG. 6. NKG7 has been reported to contain 4 transmembrane regions, confirmed for the human (A) and mouse (B) protein, by the transmembrane prediction program TMpred (https://embnet.vital-it.ch/software/TMPRED_form.html). The predicted model of the NKG7 protein reveals two extracellular loops, identified by the arrows, that exists in both human and mouse NKG7 (C).

FIG. 7. Splenic mononuclear cells from C57BL/6N (B6N; wild-type) and B6N.Nkg7^{-/-} mice were stimulated with either purified monoclonal anti-mouse (α)-CD3+α-CD28+ recombinant (r)IL-2 (Th0 condition), lipopolysaccharide (LPS), or ODN 1826 (CpG) for 24 hours. Higher levels of the anti-inflammatory cytokine, interleukin (IL)-10, were detected by cytometric bead array (CBA) in the supernatant collected from the LPS and CpG wells. n=3 (triplicate). Statistical significance determined using a two-way analysis of variance (ANOVA) with Sidak's multiple comparisons test. ** and *** indicate a p value of <0.01 and 0.001, respectively.

FIG. 8. The line graphs show the changes in proportion of GFP-expressing cells within each cell subset at the indicated time points during L. donovani infection. Statistical significance was determined using the Kruskal-Wallis test with Dunn's multiple comparisons test. * indicates p value and error bars represent mean±standard error of mean (SEM).

FIG. 9. Wild-type (WT; C57BL/6N) and Nkg7-deficient (B6N.Nkg7^{-/-}) mice were infected with L. donovani. Nkg7-deficiency resulted in significantly increased parasite burdens in the spleen and liver (expressed as Leishman-Donovan Units (LDU)) (A). Nkg7-deficient mice produced significantly less pro-inflammatory cytokines at day 14 p.i. (B). CD4⁺ T cells were purified by magnetic-activated cell sorting either from WT or Nkg7-deficient mice and transferred into Rag1^{-/-} mice. Recipient mice were subsequently infected with L. donovani. Assessment of parasite burdens in the liver at day 14 p.i. showed increased parasite burdens in the liver of Rag1^{-/-} mice that received Nkg7-deficient CD4⁺ T cells, compared to Rag1^{-/-} mice that received WT CD4⁺ T cells. This indicates that the phenotype observed in Nkg7-deficient mice is mediated at least in part by CD4⁺ T cells (C). Statistical significance was determined using a two-way analysis of variance (ANOVA) with multiple comparisons in (A and B) or Mann-Whitney test (C). p value is indicated where * p<0.05, ** p<0.01, *** p<0.001, and **** p<0.0001. Error bars represent mean±standard error of mean (SEM). Data is representative of one experiment, where n=3 or 4 biological replicates per strain, per time point.

FIG. 10. Wild-type (WT; C57BL/6N) or Nkg7-deficient (B6N.Nkg7^{-/-}) mice received either B16F10 or LWT1 cells and lungs were harvested 14 days later to determine the number of lung metastases. In both cases, Nkg7-deficient mice exhibited increased lung metastases (A). Natural killer (NK) cells were sorted from either WT or Nkg7-deficient mice and transferred into Rag2γc^{-/-} mice. Recipient mice were then injected with either 1×10⁴ or 1×10⁵ B16F10 cells. Rag2γc^{-/-} mice that received Nkg7-deficient NK cells showed increased lung metastases compared to those that received WT NK cells, consistent with the observations made in systemic Nkg7-deficient mice. This indicates that the increase in lung metastases in systemic Nkg7-deficient mice injected with B16F10 cells is mediated by NK cells (B). Statistical testing was performed using unpaired t-tests. p value is indicated where * p<0.05 and **** p<0.0001.

FIG. 11. Wild-type (WT; C57BL/6N) or Nkg7-deficient (B6N.Nkg7^{-/-}) mice were given drinking water containing dextran sodium sulfate (DSS) for 6 days. Nkg7-deficient mice were found to have a lower disease activity index (DAI) indicative of less severe disease compared to WT mice. Less weight loss (expressed as % of starting body weight) was also observed in Nkg7-deficient mice relative to WT mice (A). Nkg7-deficient mice had increased colon lengths compared to WT mice when measured 7 days after initial administration of DSS. Colon shortening is associated with increased inflammation (B). Hematoxylin and Eosin (H&E) staining of colon sections show a complete loss of crypts in WT mice at day 7 after the initial administration of DSS (C). Histological scoring of colon sections indicate that Nkg7-deficient mice had less inflammation than WT mice, both in the medial and distal sections of the colon (D). Statistical significance was determined using a two-way analysis of variance (ANOVA) with Sidak's multiple comparisons test (A and D) or an unpaired t-test (B). p value is indicated where * p<0.05, ** p<0.01, *** p<0.001, and **** p<0.0001. Error bars represent mean±standard error of mean (SEM). Data is representative of two experiments, where n=8-10 biological replicates per group.

FIG. 12. Wild-type (WT; C57BL/6N) or Nkg7-deficient (B6N.Nkg7^{-/-}) mice were infected with luciferase-expressing Plasmodium berghei ANKA (PbA). Nkg7-deficient mice showed increased levels of parasitised red blood cells (pRBC) compared to WT mice (A). Nkg7-deficient mice recorded lower experimental cerebral malaria (ECM) scores than WT mice (B). WT mice succumbed to infection between days 7 and 8 p.i, while Nkg7-deficient mice survived up to day 14 p.i. (C). Bioluminescence imaging shows increased bioluminescence and therefore parasite burdens in the whole body (D) and brain (E) of WT mice compared to Nkg7-deficient mice. Statistical testing was performed using a two-way analysis of variance (ANOVA) with Sidak's multiple comparisons test (A), the log-rank (Mantel-Cox) test (C) or a Mann-Whitney test (D and E). p value is indicated where * p<0.05 and *** p<0.001. Error bars represent mean±standard error of mean (SEM). n=5 mice per group.

### DETAILED DESCRIPTION

### General Techniques and Definitions

Unless specifically defined otherwise, technical and scientific terms used herein shall be taken to have the same meaning as commonly understood by one of ordinary skill in the art (e.g., in immunology, immunohistochemistry, protein chemistry, cell biology, biochemistry, and chemistry).

Unless otherwise indicated, the recombinant protein, cell culture, and immunological techniques utilized in the present disclosure are standard procedures, known to those skilled in the art, such as those described in J. Perbal, A Practical Guide to Molecular Cloning, John Wiley and Sons (1984), J. Sambrook et al., Molecular Cloning: A Laboratory Manual, 3rd edn, Cold Spring Harbour Laboratory Press (2001), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including updates until present).

The term “and/or”, e.g., “X and/or Y” shall be understood to mean either “X and Y” or “X or Y” and shall be taken to provide explicit support for both meanings or for either meaning.

As used herein, the term “disease” or “condition” refers to a disruption of or interference with normal function, and is not to be limited to any specific condition, disease or disorder.

As used herein, the terms “treating”, “treat”, or “treatment” include administering a compound or molecule described herein to reduce, prevent, or eliminate at least one symptom of a disease or condition.

As used herein, the terms “preventing”, “prevent”, or “prevention” include administering a therapeutically effective amount of a compound or molecule sufficient to stop or hinder the development of at least one symptom of a disease or condition.

As used herein, the term “subject” shall be taken to mean any animal including humans, for example a mammal. Exemplary subjects include but are not limited to humans, mice and rats. In one example, the subject is a human. In another example, the subject is a mouse.

“Administering” as used herein is to be construed broadly and includes administering a compound or molecule as described herein to a subject as well as providing a compound or molecule as described herein to a cell.

### NKG7

Activated natural killer (NK) cells and T cells share many cytolytic components and surface antigens. By a combination of differential and subtractive hybridization strategies, Turman et al. (1993) isolated a cDNA encoding NKG7 (OMIM entry 606008). The deduced 148-amino acid type I integral transmembrane protein has a signal sequence, a 38-residue external domain with multiple T-cell epitopes but no N-glycosylation sites or cysteine residues, and a 64-amino acid cytoplasmic domain with several phosphorylation sites.

To date, the expression of NKG7 at the protein level has been poorly described. This is primarily due to the scarcity of reagents including monoclonal antibodies and mouse models. Thus, a gap exists in the understanding of NKG7's function, the molecular mechanisms underlying its function, and the other molecules it physically interacts with. In the present disclosure, it has now been demonstrated that NKG7 is a modulator of pro-inflammatory and anti-inflammatory cytokines. Accordingly, the modulation of NKG7 activity may be used in therapeutic applications in which it is desirable to increase an immune response, for example in cancer immunotherapy and inducing an immune response to an immunogen, as well as in anti-inflammatory therapies, such as in the treatment and prevention of autoimmune disease, inflammatory conditions and graft versus host disease.

Examples of human NKG7 amino acid sequences are provided in SEQ ID NOs:1, 3 and 5, and their nucleic acid coding sequences are provided in SEQ ID NOs:2, 4 and 6. Examples of murine amino acid and nucleic acid NKG7 sequences are provided in SEQ ID Nos:7, 8, 9 and 10.

In some embodiments, NKG7 may comprises an amino acid sequence at least 80% identical to any one of SEQ ID Nos:1, 3, 5, 7, or 9. In some embodiments, NKG7 may comprise an amino acid sequence at least 85%, or at least 86%, 87%, 88%, or 89%, or at least 90%, 91%, 92%, 93%, 94%, 95%, 96%, 97%, 98%, or 99% identical to any of SEQ ID Nos:1, 3, 5, 7 or 9.

In some embodiments, NKG7 may be encoded by a nucleic acid molecule which hybridises under stringent conditions with a nucleic acid comprising any one of SEQ ID Nos:2, 4, 6, 8, or 10.

In some embodiments, NKG7 consists of a sequence selected from any one of SEQ ID Nos:1, 3, 5, 7, and 9 and having substitution of, or deletion or addition of, one or several amino acids.

‘NKG7 activity’ as used herein, refers to the ability of NKG7 to regulate the production of pro-inflammatory and/or anti-inflammatory cytokines. In particular, NKG7 activity refers to the ability of NKG7 to induce or upregulate the expression of pro-inflammatory cytokines such as interferon gamma (IFNγ), tumor necrosis factor (TNFα), and IL-6 in a subject, and/or the ability of NKG7 to inhibit or reduce the expression of anti-inflammatory cytokines.

Accordingly, the term ‘modulating NKG7 activity’ refers to either increasing or inhibiting the ability of NKG7 to induce pro-inflammatory cytokine expression and/or regulate anti-inflammatory cytokine expression, depending on the desired therapeutic outcome. In some embodiments, modulating NKG7 activity may be achieved with compounds that bind directly to NKG7, such as, for example, antibodies that bind NKG7 and modulate NKG7 activity. In some embodiments modulating NKG7 activity is achieved by using compounds that modulate expression of NKG7, for example, such as siRNA molecules, which as a result of modulating NKG7 expression also modulate NKG7 activity (for example, in some embodiments, reduced NKG7 expression results in reduced NKG7 activity in a subject).

The level of NKG7 expression and/or activity may be determined using any suitable means known in the art. For example, the level of expression and/or activity of NKG7 may be determined by immunoassay, using antibodies that bind to NKG7. Alternatively or in addition, the level of expression and/or activity of NKG7 may be determined by nucleic acid detection methods known in the art. In some embodiments, NKG7 activity is determined by measuring the level of pro-inflammatory cytokines and/or anti-inflammatory cytokines in a sample from a subject, for example, such as in a blood sample from a subject. For example, NKG7 activity may be determined by measuring the level of pro-inflammatory cytokines such as IL-6, INF-γ and TNFα, and/or measuring the level of anti-inflammatory cytokines such as IL-10, IL-4 or IL-13.

The term ‘increasing NKG7 activity’ as used herein refers to the use of compounds that are capable of upregulating NKG7 expression or activity, thus resulting in an increase in pro-inflammatory cytokines and/or a decrease in anti-inflammatory cytokines. For example, in some embodiments, the compound that increases NKG7 activity may be a small molecule, NKG7 ligand or agonist antibody that binds NKG7. In some embodiments, the compound that increases NKG7 activity may be an NKG7 polypeptide or fragment thereof having NKG7 activity.

Thus, the term “inhibiting NKG7 activity” as used herein refers to the use of compounds that are capable of decreasing or downregulating NKG7 expression or activity, thus resulting in a decrease in pro-inflammatory cytokines and/or an increase in anti-inflammatory cytokines. For example, in some embodiments, the compound that inhibits NKG7 activity may be a small molecule, NKG7 ligand or antibody that binds NKG7. In some embodiments, the compounds capable of inhibiting NKG7 activity may be nucleic acid molecules that inhibit or downregulate expression of NKG7. For example, the nucleic acid molecules may be siRNA, double-stranded RNA, antisense nucleic acids, or miRNA.

### Compounds

A “compound”, as contemplated by the present disclosure, can take any of a variety of forms including natural compounds, chemical small molecule compounds or biological compounds or macromolecules. Exemplary compounds include an antibody or an antigen binding fragment of an antibody, a nucleic acid, a polypeptide, a peptide, and a small molecule.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

The skilled artisan will be aware that an “antibody” is generally considered to be a protein that comprises a variable region made up of a plurality of polypeptide chains, e.g., a polypeptide comprising a VL and a polypeptide comprising a VH. An antibody also generally comprises constant domains, some of which can be arranged into a constant region, which includes a constant fragment or fragment crystallizable (Fc), in the case of a heavy chain. A VH and a VL interact to form a Fv comprising an antigen binding region that is capable of specifically binding to one or a few closely related antigens. Generally, a light chain from mammals is either a κ light chain or a 2 light chain and a heavy chain from mammals is α, δ, ε, γ, or μ. Antibodies can be of any type (e.g., IgG, IgE, IgM, IgD, IgA, and IgY), class (e.g., IgG1, IgG2, IgG3, IgG4, IgA1 and IgA2) or subclass. The term “antibody” also encompasses humanized antibodies, primatized antibodies, human antibodies and chimeric antibodies.

As used herein, the term “Fv” shall be taken to mean any protein, whether comprised of multiple polypeptides or a single polypeptide, in which a VL and a VH associate and form a complex having an antigen binding site, i.e., capable of specifically binding to an antigen. The VH and the VL which form the antigen binding site can be in a single polypeptide chain or in different polypeptide chains. Furthermore, an Fv of the disclosure (as well as any protein of the disclosure) may have multiple antigen binding sites which may or may not bind the same antigen. This term shall be understood to encompass fragments directly derived from an antibody as well as proteins corresponding to such a fragment produced using recombinant means. In some examples, the VH is not linked to a heavy chain constant domain (CH) 1 and/or the VL is not linked to a light chain constant domain (CL). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., CH2 or CH3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a VH and a single constant domain Two Fab′ fragments are obtained per antibody treated in this manner A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab2” fragment is a recombinant fragment comprising two Fab fragments linked using, for example a leucine zipper or a CH3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of a compound or an antigen binding site thereof with an antigen means that the interaction is dependent upon the presence of a particular structure (e.g., an antigenic determinant or epitope) on the antigen.

As used herein, the term “specifically binds” or “binds specifically” shall be taken to mean that a compound of the disclosure reacts or associates more frequently, more rapidly, with greater duration and/or with greater affinity with a particular antigen or cell expressing same than it does with alternative antigens or cells. For example, a compound binds to NKG7 with materially greater affinity (e.g., 20 fold or 40 fold or 60 fold or 80 fold to 100 fold or 150 fold or 200 fold) than it does to other cytokine receptor or to antigens commonly recognized by polyreactive natural antibodies (i.e., by naturally occurring antibodies known to bind a variety of antigens naturally found in humans)

In some embodiments, the antibody that binds NKG7 may be a multi-specific antibody, such as a bi-specific antibody, e.g. composed of two different fragments of two different antibodies, such that the bi-specific antibody binds two types of antigen. For example, a bi-specific antibody may comprise a fragment that binds NKG7, and a second fragment that binds to a second antigen. The second antigen may be, for example, a marker of an immune cell, for example such as CD56 on NK cells, or a marker specific for CD4⁺ T-cells or CD8⁺ T-cells. Techniques for the preparation of bi-specific antibodies are well known in the art.

Antibodies that bind NKG7 are commercially available. For example, Polyclonal anti-NKG7 antibody is available (HPA071454; Sigma Aldrich), and monoclonal antibody that binds NKG7 is also commercially available (anti-TIA-1 (anti-NKG-7); IM2550; Beckman Coulter).

Methods for generating and isolating antibodies that bind to a specified antigen are well known in the art.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

As used herein, the term “antigen binding site” shall be taken to mean a structure formed by a protein that is capable of binding or specifically binding to an antigen. The antigen binding site need not be a series of contiguous amino acids, or even amino acids in a single polypeptide chain. For example, in a Fv produced from two different polypeptide chains the antigen binding site is made up of a series of amino acids of a VL and a VH that interact with the antigen and that are generally, however not always in the one or more of the CDRs in each variable region. In some examples, an antigen binding site is a VH or a VL or a Fv.

As used herein, the term “epitope” (syn. “antigenic determinant”) shall be understood to mean a region of NKG7 to which a protein comprising an antigen binding site of an antibody binds. This term is not necessarily limited to the specific residues or structure to which the protein makes contact. For example, this term includes the region spanning amino acids contacted by the protein and/or 5-10 or 2-5 or 1-3 amino acids outside of this region. In some examples, the epitope comprises a series of discontinuous amino acids that are positioned close to one another when NKG7 is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

In some embodiments of the disclosure, therapeutic and/or prophylactic methods as described herein involve reducing expression of NKG7. For example, such a method may involve administering a compound that reduces transcription and/or translation of a nucleic acid encoding NKG7. In one example, the compound that inhibits NKG7 activity is a nucleic acid, e.g., an antisense polynucleotide, a ribozyme, a PNA, an interfering RNA, a siRNA, or a microRNA.

RNA interference (RNAi) is useful for specifically inhibiting the production of a particular protein. Without being limited by theory, this technology relies on the presence of dsRNA molecules that contain a sequence that is essentially identical to the mRNA of the gene of interest or part thereof, in this case an mRNA encoding NKG7. Conveniently, the dsRNA can be produced from a single promoter in a recombinant vector host cell, where the sense and anti-sense sequences are flanked by an unrelated sequence which enables the sense and anti-sense sequences to hybridize to form the dsRNA molecule with the unrelated sequence forming a loop structure, such as a short hairpin RNA (shRNA). The design and production of suitable dsRNA molecules for the present disclosure is well within the capacity of a person skilled in the art, particularly considering WO99/32619, WO99/53050, WO99/49029, and WO01/34815. Such dsRNA molecules for RNAi include, but are not limited to short hairpin RNA (shRNA) and bi-functional shRNA.

Exemplary small interfering RNA (“siRNA”) molecules comprise a nucleotide sequence that is identical to about 19-21 contiguous nucleotides of the target mRNA. For example, the siRNA sequence commences with the dinucleotide AA, comprises a GC-content of about 30-70% (for example, 30-60%, such as 40-60% for example about 45%-55%), and does not have a high percentage identity to any nucleotide sequence other than the target in the genome of the mammal in which it is to be introduced, for example as determined by standard BLAST search.

The term “antisense nucleic acid” shall be taken to mean a DNA or RNA or derivative thereof (e.g., LNA or PNA), or combination thereof that is complementary to at least a portion of a specific mRNA molecule encoding a polypeptide as described herein in any example of the disclosure and capable of interfering with a post-transcriptional event such as mRNA translation. The use of antisense methods is known in the art (see for example, Hartmann and Endres (editors), Manual of Antisense Methodology, Kluwer (1999)).

An antisense nucleic acid of the disclosure will hybridize to a target nucleic acid under physiological conditions. Antisense nucleic acids include sequences that correspond to structural genes or coding regions or to sequences that effect control over gene expression or splicing. For example, the antisense nucleic acid may correspond to the targeted coding region of a nucleic acid encoding NKG7, or the 5′-untranslated region (UTR) or the 3′-UTR or combination of these. It may be complementary in part to intron sequences, which may be spliced out during or after transcription, for example only to exon sequences of the target gene. The length of the antisense sequence should be at least 19 contiguous nucleotides, for example, at least 50 nucleotides, such as at least 100, 200, 500 or 1000 nucleotides of a nucleic acid encoding NKG7. The full-length sequence complementary to the entire gene transcript may be used. The degree of identity of the antisense sequence to the targeted transcript should be at least 90%, for example, 95-100%.

In another example, the compound may be a nucleic acid aptamer (adaptable oligomer). Aptamers are single stranded oligonucleotides or oligonucleotide analogs that are capable of forming a secondary and/or tertiary structure that provides the ability to bind to a particular target molecule, such as a protein or a small molecule, e.g., NKG7. Thus, aptamers are considered the oligonucleotide analogy to antibodies. In general, aptamers comprise about 15 to about 100 nucleotides, such as about 15 to about 40 nucleotides, for example about 20 to about 40 nucleotides, since oligonucleotides of a length that falls within these ranges can be prepared by conventional techniques.

An aptamer can be isolated from or identified from a library of aptamers. An aptamer library is produced, for example, by cloning random oligonucleotides into a vector (or an expression vector in the case of an RNA aptamer), wherein the random sequence is flanked by known sequences that provide the site of binding for PCR primers. An aptamer that provides the desired biological activity (e.g., binds specifically to NKG7) is selected. An aptamer with increased activity is selected, for example, using SELEX (Sytematic Evolution of Ligands by EXponential enrichment). Suitable methods for producing and/or screening an aptamer library are described, for example, in Elloington and Szostak, Nature 346:818-22, 1990; U.S. Pat. No. 5,270,163; and/or U.S. Pat. No. 5,475,096.

### Modulation of NKG7 Activity by Gene Editing

In some embodiments, gene editing systems may be used to modulate activity of NKG7. In one example, clustered regularly interspaced short palindromic repeats (CRISPR) and the CRISPR-associated 9 (Cas9) enzyme can be used for a technology known as CRIPR/Cas9 to edit genes within individual cells and whole organisms (Zhang et al., 2014). This technology can be used to delete NKG7 from human or animal germline DNA (Hultquist et al., 2016). Alternatively, CRISPR/Cas9 can be employed to modify the NKG7 gene or associated regulatory DNA regions to either enhance or limit gene transcription, and therefore, protein expression (Ledford, 2015). Similarly, inactive versions of Cas9 can be used with CRISPR to avoid cutting DNA, but instead binding to gene or RNA targets to silence genes (Dominguez, 2016). The skilled person could also employ other gene systems or technologies as known in the art to modify activity of NKG7. Such systems and technologies include nuclease-based platforms, including zinc finger nucleases, transcription activator-like effector nucleases (TALENs), and meganucleases.

In some embodiments, the activity or expression of NKG7 may be modulated in cells in vitro using a gene editing system.

In other embodiments, it may be desirable to perform an ex vivo method of modulating NKG7 activity or expression in cells that have been obtained from a subject. These modified cells may then be returned to the patient for therapeutic activity by modulating the activity or expression of NKG7 in the subject.

In another embodiment, the person skilled in the art may use a gene editing system administered to the subject to modify the activity or expression of NKG7 in the subject in vivo. Techniques known in the art for in delivery of gene editing systems include packing formats such as viral packaging, mRNA, plasmid and protein-based approaches.

### Screening Assays

In light of the present disclosure, the person skilled in the art will be able to conduct screening assays to identify antibodies and molecules that bind to and/or modulate the activity of NKG7, including the assays described herein in the Examples section. For example, the skilled person can perform T-cell activation assays in the presence of a test compound according to the method described in Example 1. Thus, the assay may involve the activation of a purified population of mouse or human T-cells, mouse splenocytes or human PBMCs with a stimulus such as CPG or LPS, and detecting an increase or inhibition of cytokine production. As understood in the art, T-cell activation assays may utilise techniques such as limiting dilutions culture, ELISPOT, cytokine capture and biosensor assays, as well as commercially available T-cell activation assays. Other assays may be based on detecting an altered capacity of APCs to present antigen to T-cells, or the capacity of T-helper cells to produce cytokines and/or modulate the activity of other immune cells such as B-cells, cytotoxic T-cells or antigen presenting cells.

### Treatment and Prevention of Autoimmune Disease and Inflammatory Conditions

Disclosed herein are therapeutic methods in which the inhibition or reduction of expression of pro-inflammatory cytokines and/or an increase in anti-inflammatory cytokines is therapeutically beneficial, such as in autoimmunity, inflammation, allergic disorders, sepsis, GVHD or in alleviating the inflammatory side effects of other disease conditions. Thus, in some embodiments, compounds that inhibit NKG7 activity may be administered to a subject for the treatment or prevention of autoimmune disease and/or inflammatory conditions.

“Autoimmunity” or “autoimmune disease or condition” as used herein, refers broadly to a disease or disorder arising from and directed against an individual's own tissues or a co-segregate or manifestation thereof or resulting condition therefrom, and includes. Herein autoimmune conditions include inflammatory or allergic conditions, e.g., chronic diseases characterized by a host immune reaction against self-antigens potentially associated with tissue destruction such as rheumatoid arthritis.

In some embodiments, the autoimmune disease is selected from rheumatoid arthritis, type-I diabetes, multiple sclerosis, Hashinoto's thyroiditis, systemic lupus erythromatosus, gastritis, autoimmune hepatitis, hemolytic anemia, autoimmune hemophilia, autoimmune lymphoproliferative syndrome (ALPS), autoimmune uveoretinitis, glomerulonephritis, Guillain-Barre syndrome, psoriasis, alopecia, and myasthenia gravis.

“Inflammatory disorders”, “inflammatory conditions” and/or “inflammation”, used interchangeably herein, refers broadly to chronic or acute inflammatory diseases, and expressly includes inflammatory autoimmune diseases and inflammatory allergic conditions. These conditions include by way of example inflammatory abnormalities characterized by dysregulated immune response to harmful stimuli, such as pathogens, damaged cells, or irritants. Inflammatory disorders underlie a vast variety of human diseases. Diseases with etiological origins in inflammatory processes include cancer, atherosclerosis, neuroinflammation and ischemic heart disease. Examples of disorders associated with inflammation include: chronic prostatitis, glomerulonephritis, Hypersensitivities, Pelvic inflammatory disease, Reperfusion injury, Sarcoidosis, Vasculitis, Interstitial cystitis, normocomplementemic urticarial vasculitis, pericarditis, myositis, anti-synthetase syndrome, scleritis, macrophage activation syndrome, Behcet's Syndrome, PAPA Syndrome, Blau's Syndrome, gout, adult and juvenile Still's disease, cryropyrinopathy, Muckle-Wells syndrome, familial cold-induced auto-inflammatory syndrome, neonatal onset multisystemic inflammatory disease, familial Mediterranean fever, chronic infantile neurologic, cutaneous and articular syndrome, systemic juvenile idiopathic arthritis, Hyper IgD syndrome, Schnitzler's syndrome, TNF receptor-associated periodic syndrome (TRAPSP), gingivitis, periodontitis, hepatitis, cirrhosis, pancreatitis, myocarditis, vasculitis, gastritis, gout, gouty arthritis, and inflammatory skin disorders, selected from the group consisting of psoriasis, atopic dermatitis, eczema, rosacea, urticaria, and acne; and other conditions including systemic inflammatory response syndrome (SIRS), Alzheimer's disease, and Parkinson's disease.

In some embodiments, the autoimmune and/or inflammatory condition is an inflammatory bowel disorder. As used herein, the term “inflammatory bowel disorder”, includes a disorder that is diagnosed by at least one of the following criteria 1) inflammation in the wall of the colon and/or 2) a stool sample that tests positive for inflammatory markers of inflammation in combination with a patient that complains of at least one symptom. The symptoms include but are not limited to low abdominal pain relieved by defecation, alternating constipation/diarrhoea, passage of small calibre stools, cramping, diarrhoea, constipation, urgency, flatulence, watery diarrhoea with or without pain, distension, nausea and or incontinence. Inflammatory bowel disorder includes inflammatory irritable bowel syndrome, Crohn's disease, irritable bowel syndrome-diarrhoea, irritable bowel syndrome-constipation, irritable bowel syndrome-mixed, irritable bowel syndrome-alternating, dyspepsia, microscopic colitis, lymphocytic colitis, collagenous colitis, indeterminate colitis, celiac disease, and combinations thereof.

In some embodiments, the inflammatory bowel disorder is inflammatory bowel disease (IBD). Inflammatory bowel disease includes both Crohn's disease and ulcerative colitis. Crohn's disease is an inflammatory bowel disease that may affect any part of the gastrointestinal tract from the mouth to the anus. It causes inflammation of the lining of the gastrointestinal tract, which can lead to abdominal pain, severe diarrhoea, fatigue, weight loss, and malnutrition. Inflammation caused by Crohn's disease can involve different areas of the gastrointestinal tract in different people. The inflammation associated with Crohn's disease often spreads deep into the layers of the affected bowel tissue. Crohn's can be both painful and debilitating, and sometimes may lead to life-threatening complications. Complications include intestinal blockages, ulcers in the intestine, and problems obtaining sufficient nutrients. Other complications occur outside the gastrointestinal tract and include anemia, skin rashes, arthritis, inflammation of the eye, and tiredness. Conventional treatment may help to control symptoms, and includes medicines, nutritional supplements, and/or surgery. While some patients may have long periods of remission, in which they are free of symptoms, there is currently no cure for Crohn's disease. Accordingly, there remains a need for methods for the management and treatment of Crohn's disease.

Ulcerative colitis is a chronic disease of the large intestine, also known as the colon, in which the lining of the colon becomes inflamed and develops tiny open sores, or ulcers, that produce pus and mucous. The combination of inflammation and ulceration can cause abdominal discomfort and frequent emptying of the colon. Existing treatments for ulcerative colitis involve intense and lengthy combinational drug therapy with significant side effects or even require surgery to remove part of the colon. Thus, there is a need for more effective treatments for ulcerative colitis that are easier to administer and that can cure this debilitating condition.

In some embodiments, the present disclosure provides a method of treating or preventing graft-versus-host disease in recipients of transplants, particularly allogeneic transplants. GVHD is a common complication following an allogeneic tissue transplant. It is commonly associated with stem cell or bone marrow transplant but the term also applies to other forms of tissue graft Immune cells (white blood cells) in the tissue (the graft) recognise the recipient (the host) as “foreign”. In one embodiment, the GVHD is acute GVHD. The acute or fulminant form of the disease (aGVHD) is normally observed within the first 100 days post-transplant and is a major challenge to transplants owing to associated morbidity and mortality. Acute graft-versus-host-disease may be characterized by selective damage to the liver, skin (rash), mucosa, and the gastrointestinal tract. Newer research indicates that other graft-versus-host-disease target organs include the immune system (the hematopoietic system, e.g., the bone marrow and the thymus) itself, and the lungs in the form of idiopathic pneumonitis. Acute GVHD is staged and graded (0-IV) by the number and extent of organ involvement. Patients with grade IV GVHD usually have a poor prognosis. If the GVHD is severe and requires intense immunosuppression involving steroids and additional agents to get under control, the patient may develop severe infections as a result of the immunosuppression and may die of infection.

### Cancer Immunotherapy

The present disclosure provides a method of treating cancer in a subject by modulating the subject's immune response to cancer cells by modulating activity of NKG7. Thus, in some embodiments, treating a patient with a modulator of NKG7 may be used as a form of cancer immunotherapy. As known in the art, cancer immunotherapy is a therapy used to treat patients that involves or uses components of the immune system. Thus, in some embodiments, administering a compound to increase NKG7 activity in a patient can be used to increase an immune response against cancer cells in the patient.

In some embodiments, the methods of treatment disclosed herein may be used in combination or in conjunction with other cancer immunotherapies and immunotherapy agents.

Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines.

Among these, multiple antibody therapies are approved in various jurisdictions to treat a wide range of cancers. Cell surface receptors are common targets for antibody therapies and include CD20, CD274 and CD279. Once bound to a cancer antigen, antibodies can induce antibody-dependent cell-mediated cytotoxicity, activate the complement system, or prevent a receptor from interacting with its ligand, all of which can lead to cell death. Approved antibodies include alemtuzumab, ipilimumab, nivolumab, ofatumumab and rituximab.

Active cellular therapies usually involve the removal of immune cells from the blood or from a tumor. Those specific for the tumor are cultured and returned to the patient where they attack the tumor; alternatively, immune cells can be genetically engineered to express a tumor-specific receptor, cultured and returned to the patient. Cell types that can be used in this way are natural killer cells, lymphokine-activated killer cells, cytotoxic T cells and dendritic cells. One specific type of cellular therapy is CAR-T therapy. Chimeric antigen receptors (CARs, also known as chimeric immunoreceptors, chimeric T cell receptors or artificial T cell receptors) are engineered receptors which graft an arbitrary specificity onto an immune effector cell. Typically, these receptors are used to graft the specificity of a monoclonal antibody onto a T cell, with transfer of their coding sequence facilitated by retroviral vectors. The receptors are called chimeric because they are composed of parts from different sources. The basic principle of CAR T-Cell design involves recombinant receptors that combine antigen-binding and T-Cell activating functions. The general premise of CAR T-Cells is to rapidly generate T-Cells targeted to specific tumour cells. Scientists can remove T-cells from a patient, genetically engineer them, and then put them back into the patient to target cancer cells.

In some embodiments, the cancer immunotherapy agent may be selected from one or more of an immune checkpoint modulatory agent, a cancer vaccine, an oncolytic virus, a cytokine, and a cell-based immunotherapies.

In some embodiments, the inhibitory immune checkpoint molecule is selected from one or more of Programmed Death-Ligand 1 (PD-L1), Programmed Death 1 (PD-1), Programmed Death-Ligand 2 (PD-L2), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), V-domain Ig suppressor of T cell activation (VISTA), B and T Lymphocyte Attenuator (BTLA), CD160, Herpes Virus Entry Mediator (HVEM), and T-cell immunoreceptor with Ig and ITIM domains (TIGIT).

In some embodiments, the present disclosure provides a method of increasing, enhancing or stimulating an immune response or function in an individual having cancer comprising administering to the individual an effective amount of a compound that increases NKG7 activity and an anti-cancer agent or an anti-cancer therapy. In other aspects, the present disclosure provides use of an effective amount of an agent that increases NKG7 activity in the manufacture of a medicament for increasing, enhancing or stimulating an immune response or function in an individual having cancer, wherein the agent that increases NKG7 activity is used in combination with an anti-cancer agent or an anti-cancer therapy. In other aspects, the present disclosure provides use of an effective amount of an anti-cancer agent in the manufacture of a medicament for increasing, enhancing or stimulating an immune response or function in an individual having cancer, wherein the anti-cancer agent is used in combination with a compound that increases NKG7 activity. In other aspects, the present disclosure provides a pharmaceutical composition comprising an agent that increases NKG7 activity for use in increasing, enhancing or stimulating an immune response or function in combination with an anti-cancer agent or an anti-cancer therapy. In other aspects, the present disclosure provides a pharmaceutical composition comprising an anti-cancer agent for use in increasing, enhancing or stimulating an immune response or function in combination with an agent that increases NKG7 activity.

In some embodiments, a compound that increases NKG7 may be used in combination or in conjunction with a cancer vaccine comprising a tumor associated antigen. In some embodiments, the cancer vaccine is selected from one or more of Oncophage, a human papillomavirus HPV vaccine optionally Gardasil or Cervarix, a hepatitis B vaccine optionally Engerix-B, Recombivax HB, or Twinrix, and sipuleucel-T (Provenge), or comprises a cancer antigen selected from one or more of human Her2/neu, Her1/EGF receptor (EGFR), Her3, A33 antigen, B7H3, CD5, CD19, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin .alpha.v.beta.3, integrin .alpha.5.beta.1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin.

In some embodiments, the compound that increases activity of NKG7 and the cancer immunotherapy agent are administered separately to the subject. In some embodiments, the compounds that increases activity of NKG7 and the cancer immunotherapy agent are administered together as part of the same composition.

It will be appreciated that cancer immunotherapy methods involving the use of compounds increase activity of NKG7 may be performed in isolation or as an adjunct to other known cancer therapy regimes. For example, treatment may be conducted in conjunction with or after treatments such as chemotherapy, radiation therapy, stem cell transplant and/or immunotherapy, for example, monoclonal antibody therapy. Examples of chemotherapeutic agents used in the treatment of brain tumors include temozolomide, BCNU (Carmustine), PCV (combination of procarbazine, CCNV (Lomustine), and vincristine), carboplatin, etoposide, irinotecan, Cis-Retonoic acid, thalidomide, tamoxifen and COX-2 inhibitors. Other known chemotherapeutic agents include chlorambucil, cyclophosphamide, melphalan, daunorubicin, doxorubicin, idarubicin, mitoxantrone, methotrexate, fludarabine, cytarabine, etoposide, topotecan, prednisone, dexamethasone, vincristine and vinblastine.

### Infectious Diseases and Vaccination

In some embodiments, the present disclosure provides methods of increasing or enhancing an immune response in the treatment or prevention of an infectious disease and/or enhancing an immune response to a vaccine antigen from an infectious agent.

As used herein, a “vaccine” refers to any composition containing an immunogenic determinant (i.e. an antigen) which stimulates the immune system in a manner such that it can better respond to subsequent challenges or pathogenic infections or a tumour. It will be appreciated that a vaccine usually contains an immunogenic determinant and optionally an adjuvant, the adjuvant serving to non-specifically enhance the immune response to the immunogenic determinant

Thus, in some embodiments, the present disclosure provides a method of enhancing an immune response to a vaccine antigen in a subject by administering to the subject a composition that increases NKG7 activity. For example, by increasing NKG7 activity an enhanced immune response to an antigen can be achieved due to an increase in pro-inflammatory cytokines such as IL-6, IFN-γ and TNF-α. Thus, in some embodiments, compounds that increase NKG7 activity have an adjuvant effect when administered in combination and or in conjunction with a vaccine or vaccine antigen.

In some embodiments, compounds that increase NKG7 activity may be used to enhance an immune response to vaccine antigens from bacterial, viral and/or parasitic pathogens.

In some embodiments, the present disclosure provides a method of reducing an inflammatory immune response caused by an infectious agent. For example, it may be desirable to reduce an inflammatory immune response in a subject suffering from a systemic inflammatory immune response and/or it may be desirable to reduce an inflammatory immune response in a patient with sepsis. The inflammatory immune response may be reduced by administering to the subject a compound that inhibits NKG7 expression or activity.

### Compositions

Compositions comprising a compound that modulates NKG7 activity together with an acceptable carrier or diluent are useful in the methods disclosed herein. Therapeutic compositions can be prepared by mixing the desired compounds having the appropriate degree of purity with optional pharmaceutically acceptable carriers, excipients, or stabilizers (Remington's Pharmaceutical Sciences, 16th edition, Osol, A. ed. (1980)), in the form of lyophilized formulations, aqueous solutions or aqueous suspensions. Acceptable carriers, excipients, or stabilizers are preferably nontoxic to recipients at the dosages and concentrations employed, and include buffers such as Tris, HEPES, PIPES, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, colloidal silica, magnesium trisilicate, polyvinyl pyrrolidone, and cellulose-based substances.

Therapeutic compositions to be used for in vivo administration should be sterile. This is readily accomplished by filtration through sterile filtration membranes, prior to or following lyophilization and reconstitution. The composition may be stored in lyophilized form or in solution if administered systemically. If in lyophilized form, it is typically formulated in combination with other ingredients for reconstitution with an appropriate diluent at the time for use. An example of a liquid formulation is a sterile, clear, colourless unpreserved solution filled in a single-dose vial for subcutaneous injection.

Single or multiple administrations of the compositions are administered depending on the dosage and frequency as required and tolerated by the patient. The dosage and frequency will typically vary according to factors specific for each patient depending on the specific therapeutic or prophylactic agents administered, the severity and type of disease or condition, the route of administration, as well as age, body weight, response, and the past medical history of the patient. Suitable regimens can be selected by one skilled in the art by considering such factors and by following, for example, dosages reported in the literature and recommended in the Physician's Desk Reference (56th ed., 2002). Generally, the dose is sufficient to treat or ameliorate symptoms or signs of disease without producing unacceptable toxicity to the patient.

In any treatment regimen, the therapeutic composition may be administered to a patient either singly or in a combination containing other therapeutic agents, compositions, or the like, including, but not limited to, immunosuppressive agents, tolerance-inducing agents, potentiators and side-effect relieving agents. Examples of immunosuppressive agents include prednisone, melphalain, prednisolone, DECADRON (Merck, Sharp & Dohme, West Point, Pa.), cyclophosphamide, cyclosporine, 6-mercaptopurine, methotrexate, azathioprine and i.v. gamma globulin or their combination. Preferred potentiators include monensin, ammonium chloride, perhexiline, verapamil, amantadine and chloroquine. All of these agents are administered in generally accepted efficacious dose ranges such as those disclosed in the Physician's Desk Reference, 41st Ed., Publisher Edward R. Barnhart, N.J. (1987).

### EXAMPLES

### Example 1. Methods and Materials

### Mice

Female mice aged between 8-12 weeks old were used. C57BL/6J (wildtype; WT) mice were purchased from the Walter and Eliza Hall Institute (WEHI; Kew, VIC, Australia). C57BL/6N and B6N.Nkg7-KO mice were bred in-house. All mice were housed under pathogen-free conditions at the QIMR Berghofer Medical Research Institute Animal Facility (Herston, QLD, Australia). Experimental use was in accordance with the “Australian Code of Practice for the Care and Use of Animals for Scientific Purposes” (Australian National Health and Medical Research Council) and approved by the QIMR Berghofer Medical Research Institute Animal Ethics Committee (Herston, QLD, Australia; approval number: A02-633M, A02-634M, or A1707615M).

Generation of Nkg7-cre×B6.mT/mG Mice

C57BL/6J mice expressing the cre recombinase under the control of the Nkg7 promoter (B6J.Nkg7-cre) were generated by the Melbourne Advanced Genome Editing Centre (MAGEC) at the Walter and Eliza Hall Institute (WEHI) using CRISPR/Cas9 mediated gene editing. Briefly, based on methods previously described, the single guide (sg)RNA (sequence: CATGGAGCCCTGCCGGTCCC) was used to induce double stranded breaks in the Nkg7 locus to stimulate homologous recombination and a targeting vector containing ˜2 kb homology arms was used to introduce the cre recombinase coding sequence.

Forward (ACGACCAAGTGACAGCAATG) and reverse (GCTAACCAGCGTTTTCGTTC) primers to detect the cre recombinase sequence were used to screen viable pups for integration of the targeting vector by polymerase chain reaction (PCR). A 301 bp amplicon was detected where the cre recombinase sequence was present. F0 mice expressing the cre sequence were selected for backcrossing that resulted in heterozygous F1 mice. The PCR described above was used to screen F1 mice for the cre sequence. Further validation by long-range PCR was performed to verify correct positional integration of the targeting vector.

B6J.Nkg7-cre mice were crossed to mT/mG (B6.129(Cg)-Gt(ROSA)26Sor^{tm4(ACTB-tdTomato,-EGFP)Luo}/J) mice once to generate an Nkg7 reporter strain (Nkg7-cre.mT/mG). B6N.Nkg7^{-/-} (Nkg7^{tm1.1(KOMP)Vlcg}) mice were generated by the University of California Davis (UC Davis, Davis Calif., USA) as part of the trans-NIH Knockout Mouse Project (KOMP) and obtained from the KOMP Repository (http://www.komp.org/).

### Leishmania Infection in Mice

L. donovani parasites (Ethiopian strain, Lv9, MHOM/ET/67/HU3) was maintained by passage in Rag1^{-/-} mice. Passage mice were euthanised and the spleen was excised into 5 ml of sterile Roswell Park Memorial Institute Medium (RPMI; Life Technologies, Carlsbad Calif., U.S.A.)+100 μg/ml penicillin and streptomycin (PS; Gibco®, Life Techonologies); RPMI/PS) medium. The excised spleen was homogenised using a glass tissue grinder and the cell suspension was centrifuged at 115×g for 5 minutes at room temperature, with brakes off. The supernatant was transferred to a new tube, and the pellet discarded. The supernatant was centrifuged at 1960×g for 15 minutes at room temperature. The supernatant was discarded, and the pellet was incubated for 5 minutes in 1 ml of red blood cell lysing buffer Hybri-Max™ (Sigma-Aldrich®, St. Louis Mo., U.S.A.), following which, sterile RPMI/PS was added and the parasites centrifuged at 1960×g for 15 minutes at room temperature. After discarding the supernatant, sterile RPMI/PS was added to the pellet and the centrifugation step was repeated at 1960×g for 15 minutes at room temperature. After discarding the supernatant, the parasite pellet was resuspended in sterile RPMI/PS. The parasite suspension was taken up through a 26G×1½″ needle on a 1 ml syringe (Terumo®, Somerset N.J., U.S.A.) and dispensed repeatedly until a homogenous suspension was achieved. 2 μl of the parasite suspension was loaded onto a Thoma cell counting chamber (Weber Scientific International, West Sussex, U.K.) and parasites were counted in the 4×4 grid in triplicate. An average count was used to determine the number of parasites/ml using the following equation:

$$\frac{aver a g e}{16} \times 2 \times 1 {0}^{7} = parasites / ml$$

### Preparation of Spleen Single Cell Suspensions

Mice were sacrificed by CO₂ asphyxiation. A mid-sagittal incision was made on the abdominal cavity. The spleen was excised, weighed, and collected into RPMI/PS medium. Spleens were mechanically passed through an EASYstrainer™ 100 μm sterile filter (Greiner Bio-One, Kremsmunster Austria) using the back of a 5 cc/mL syringe plunger (Terumo® Medical). Cells were resuspended in RPMI/PS and centrifuged at 350×g in an Eppendorf Centrifuge 5810 R (Fisher Scientific, ThermoFisher Scientific) and lysed by incubating in Red Blood Cell Lysis Buffer Hybri-Max™ (Sigma-Aldrich®) for 7 minutes at room temperature. Cells diluted in DPBS (1×) (Gibco) and Trypan Blue Stain (Invitrogen™) and counted using the Countess II FL (Invitrogen™) as per manufacturer's protocol.

### Preparation of Liver Single Cell Suspensions

Mice were sacrificed by CO₂ asphyxiation. A mid-sagittal incision was made on the abdominal cavity. The liver was perfused with 1×phosphate-buffered saline (PBS). The excised liver was weighed and collected in 1% (v/v) FBS in PBS and mechanically passed through an EASYstrainer™ 100 μm sterile filter (Greiner Bio-One). The homogenized liver was washed twice in 1×PBS by centrifuging at 390 g in an Eppendorf Centrifuge 5810 R (Fisher Scientific, ThermoFisher Scientific). Hepatocytes were separated from lymphocytes and removed using a 33% (v/v) Percoll™ Density Gradient Media (GE Healthcare, Little Chalfont, U.K.) and centrifugation at 575 g for 15 minutes at room temperature with the brake off. Red Blood Cell Lysing Buffer Hybri-Max™ (Sigma-Aldrich®) was added to each pellet and incubated for 7 minutes at room temperature. This was followed by a single wash in 1×PBS as described above. Cells diluted in DPBS (1×) (Gibco®, Life Technologies™) and Trypan Blue Stain (Invitrogen™) and counted using the Countess II FL (Invitrogen™), as per manufacturer's protocol.

### CD4⁺ T-Cell Transfer

CD4⁺ T cells were isolated from donor mice by magnetic-activated cell sorting (MACS) using the CD4⁺ T Cell Isolation Kit, mouse, and LS columns (Miltenyi Biotec, Bergisch Gladbach, Germany) according to manufacturer's instructions. Isolated cells were diluted to 5×10⁶ cells/ml. 200 μl of cell suspension was injected i.v. into each recipient Rag1-KO mouse (1×10⁶ cells per mouse).

### Determining Parasite Burden in Murine Spleen or Liver Tissue by RT-qPCR

A piece of tissue approximately 10-20 mg was taken from the liver and spleen of each mouse post-mortem using a dermal curette blade. Tissue was collected into 150 μl of QuickExtract™ DNA Extraction Solution (Epicentre, Madison Wis., U.S.A.) and incubated at 65° C. for 30 minutes, vortexed, and incubated at 98° C. for 16 minutes to extract DNA. The amount of DNA in each sample was quantified using the Qubit® dsDNA High Sensitivity (HS) Assay Kit on a Qubit 2.0 Fluorometer (Invitrogen™) as per manufacturer's instructions. Samples were diluted to 20 ng/μl (spleen) or 7.5 ng/μl (liver) in UltraPure™ DNase/RNase-Free Distilled Water (Invitrogen™). A reaction master mix containing L. donovani GP63-specific primers and a TaqMan MGB probe, 5′-FAM-labelled reporter dye, non-fluorescent quencher previously described by Khare, et. al. (2016), and another containing the mouse Gapdh TaqMan probe was prepared using the GoTaq® Probe qPCR Master Mix (Promega, Madison Wis., U.S.A.) according to manufacturer's instructions. A total reaction volume of 10 μl containing 6 μl of reaction master mix and 4 μl of template DNA was run using the manufacturer's recommended program settings for the GoTaq® Probe qPCR Master mix, on the QuantStudio 5 Real-Time PCR System (Applied Biosystems, Foster City Calif., U.S.A.). Parasite burden was determined as a fold-change of the Ct value for L. donovani GP63 over mouse Gapdh and expressed as 2^{-ΔCT} (Schmittgen et al., 2008).

### Flow Cytometry

All flow cytometry staining was performed in Falcon® 96-Well Clear Round Bottom Tissue Culture (TC)-Treated Cell Culture Microplates (Corning Inc., Corning N.Y., U.S.A.). Single cell suspensions were incubated with 50 μl of TruStain fcX™ (anti-mouse CD16/32; clone: 93) and Zombie Aqua™ Fixable Viability Dye cocktail (both from BioLegend, San Diego Calif., U.S.A.) for 15 minutes at room temperature. Cells were washed once with staining buffer (1×PBS, 0.02% (v/v) FBS, 5 mM ethylenediaminetetraacetic acid (EDTA), 0.01% (w/v) NaN₃) by centrifuging in an Eppendorf Centrifuge 5810 R (Fisher Scientific, ThermoFisher Scientific) at 575 g for 1 minute at 4° C. Samples were then incubated with 50 μl of a cocktail of fluorescence-conjugated antibodies containing peridinin chlorophyll protein complex (PerCP)-cyanine (Cy)5.5-conjugated anti-mouse CD11b (clone: M1-70), allophycocyanin (APC)-conjugated anti-mouse TCRγδ (GL3), Alexa Fluor® 700-conjugated anti-mouse CD8a (53-6.7), APC-Cy7-conjugated anti-mouse NK1.1 (PK136), Pacific Blue-conjugated anti-mouse I-A/I-E (M5.114-15.3), Brilliant Violet™ 605-conjugated anti-mouse Ly6C (HK1.4), Brilliant Violet™ 650-conjugated anti-mouse/human B220 (RA3-6B2), Brilliant Violet™ 785-conjugated anti-mouse CD11c (N418), and phycoerythrin (PE)-Cy7-conjugated anti-mouse F4/80 (BM8) (all from Biolegend, San Diego Calif., U.S.A.) and Brilliant Ultraviolet™ 395-conjugated anti-mouse CD4 (GK1.5) and Brilliant Ultraviolet™ 737-conjugated anti-mouse TCRβ (from BD Biosciences, San Diego Calif., U.S.A.) for 30 minutes. After two washes with staining buffer, as described above, samples were incubated with 100 μl of fixation buffer from either the BD Cytofix™ Fixation Buffer Set (for cells that are subsequently stained with antibodies towards cytokines) for 20 minutes or the BD Pharmingen™ Transcription Factor Buffer Set (for cells that are subsequently stained with antibodies towards transcription factors) for 30 minutes (both from BD Biosciences). Cells were then washed twice with Perm/Wash™ Buffers from the respective kits, by centrifuging at 575 g for 1 minutes at 4° C., following which, cells were incubated with 50 μl of cocktail containing fluorescence-conjugated antibodies towards intracellular molecules for 35 minutes. All staining was performed at room temperature, in the dark. Samples were resuspended in 1% (w/v) PFA post-staining and stored at 4° C. before acquisition on a BD LSRFortessa™ (special order research product; BD Biosciences) through BD FACSDiva™ V8.0, and analysed on FlowJo v10 OSX (FlowJo, LLC, Ashland OR, U.S.A.). Graphing and statistical analyses were performed on GraphPad Prism 7 (GraphPad Software, La Jolla Calif., U.S.A.). A p value <0.05 was considered statistically significant.

t-Distributed Stochastic Neighbour Embedding (tSNE)

tSNE was performed as outlined in a protocol and R script written by Ashurst. Briefly, samples were gated on the last common population using FlowJo v10 OSX (FlowJo, LLC). Biexponential scales were then applied for all fluorophores of interest. Scales were adjusted by increasing the width basis to reduce the spread of negative data. The gated population for each sample was assigned a sample number and down-sampled on to 50,000 events on FlowJo v10 OSX (FlowJo, LLC). Down-sampled populations from all samples were then concatenated into a single file. Markers of interest were selected as tSNE parameters and the following settings were applied:
Iteration: 1000
Perplexity: 30
Eta (learning rate) 200
Theta: 0.5

Each sample within the concatenated file was distinguished on the basis of the previously assigned sample number and the tSNE parameters observed for each sample. Colourised tSNE plots were generated in R using exported channel values as an input to the script written by Ashurst (2017).

### Cytometry Bead Array (CBA)

Cytokine levels were assessed using the BD Cytometric Bead Array (CBA) Mouse Inflammation Kit or Mouse Th1/Th2/Th17 Cytokine Kit (BD Biosciences) as per manufacturer's instructions. Serum or plasma samples from mouse blood was used neat while supernatants were diluted 1:5 in 1×PBS for the detection of most cytokines. Supernatants were diluted 1:50 in 1×PBS for the detection of IFNγ. CBA data was analysed using the BD™ CBA FCAP Array Software v3.0 (BD Biosciences).

### Microarray

FACS™-sorted mouse spleen and liver CD4⁺ T cells, stored in buffer RLT, were homogenized in QIAshredder columns prior to RNA extraction using the RNeasy Mini Kit (All from QIAGEN®, Hilden, Germany) according to manufacturer's instructions. Samples were run using the Mouse Whole-Genome (WG)-6 v2.0 Expression BeadChip Kit (Illumina, San Diego Calif., U.S.A.). Quality control was assessed using the Lumi package (Du et al. 2008), run on R (https://www.r-project.org/). Differential gene expression was analysed using Limma (Ritchie et al. 2015).

Isolation of CD4⁺ T Cells from Human Peripheral Blood Mononuclear Cells (PBMCs)

Blood was obtained from symptomatic patients who were diagnosed with VL at the Kala-azar Medical Research Center (Muzaffapur, Bihar, India). Patients were diagnosed either through the detection of amastigotes in splenic aspirate smears by microscopy, or by using the rk39 dipstick test. Approximately 5 ml of blood was collected per patient at day of admission (day 0) and 30 days after treatment with AmBisome (Gilead Sciences, Inc., Foster City Calif., U.S.A.) (day 30) in a BD Vacutainer® Lithium Heparin” (LH) 170 I.U. Plus Blood Collection Tubes (BD Biosciences). Blood was layered over Ficoll-Paque™ PLUS (GE Healthcare) to isolate PBMCs. PBMCs were counted using a hemocytometer. CD4⁺ T cells were enriched by magnetic activated cell sorting (MACS) using the CD4 MicroBeads, human (Miltenyi Biotec, Bergisch Gladback, Germany) according to manufacturer's instructions.

### RNA-Sequencing

Cells were homogenized in QIAshredder columns prior to RNA extraction using the RNeasy Mini Kit (both from QIAGEN®) according to manufacturer's instructions. mRNA was isolated using the NEBNext® Poly(A) mRNA Magnetic Isolation Module (New England Biolabs Inc., Ipswich Mass., U.S.A.). Libraries were prepared using the NEBNext® Ultra™ RNA Library Prep Kit for Illumina® (New England Biolabs Inc., Ipswich Mass., U.S.A.). Libraries were quantified using the KAPA Library Quantification Kit (Roche Sequencing, Pleasanton Calif., U.S.A.) and RNA integrity number (RIN) obtained using the RNA 6000 Pico Kit (Agilent Technologies, Santa Clara Calif., U.S.A.). Expression profiling was performed by 50 bp single-end mRNA-sequencing on the Illumina HiSeq platform (performed by the Australian Genome Research Facility (AGRF), Parkville VIC, Australia).

### Bioinformatics Analysis

RNA-seq data was processed on the Galaxy platform (https://galaxy-qld.genome.edu.au/galaxy/) [7]. Quality control was performed using FastQC (Andrews, 2014). Reads were mapped using Spliced Transcripts Alignment to a Reference (STAR) (Dobin et al. 2013) and Cufflinks (Trapnell et al. 2010) was used for transcript assembly and estimating reads per kilobase of transcript per million mapped reads (RPKM). Where applicable, Cuffmerge was used to merge multiple transcript assemblies (Trapnell et al. 2012). HTseq was used to transform mapped reads to count data that was used an input for downstream analysis (Anders et al. 2015). EdgeR (Robinson et al. 2010) run on R (R Core Team, 2017, R Foundation for Statistical Computing: Vienna, Austria) was used to generate a list of differentially expressed genes from the RNA-seq dataset. Upstream regulators for genes of interest were identified using Ingenuity Pathway Analysis (IPA; QIAGEN®). In addition to drugs that were predicted on IPA, genes of interest were submitted to the Drug-Gene Interaction Database (DGIdb) (Wagner et al. 2016) to identify currently available drugs. The International Mouse Strain Resource (IMSR) was used in search of transgenic or knockout mouse strains that are available for each target gene. Localisation of target genes were determined by functional annotation based on data from Gene Ontology: Cellular Component using the Database for Annotation, Visualisation and Integrated Discovery (DAVID; v6.8 Beta, National Institute of Allergy and Infectious Diseases (NIAID), National Institutes of Health (NIH), Maryland, U.S.A.). For genes without known protein tertiary structures, the human amino acid sequence and topology was obtained from neXtProt (Beta) [20]. The human amino acid sequence was then submitted to the National Center for Biotechnology Information (NCBI) Conserved Domain Search to identify conserved domains and their protein superfamilies Gene names were entered into STRING v10.0 (Szklarczyk et al. 2015) to identify protein-protein interactions based on the gene neighbourhood, gene fusion, gene occurrence, gene coexpression, experiments, and annotated pathways, resulting in a combined score. This score served as the input into Circos Table Viewer v0.63-9 (Krzywinski et al. 2009). Graphs were plotted using Prism 7 (GraphPad Software).

### Survival and Correlation Analysis of the Cancer Genome Atlas (TCGA) Dataset

RNA-seq data previously published by the TCGA Research Network (https://cancergenome.nih.gov/) was downloaded and processed using the TCGAbiolinks package (Colaprico et al. 2016) on R. Briefly, transcript counts were normalized by gene length and genes in the bottom quartile (<25%) of expression mean across all samples were filtered out. Samples were ranked according to expression values for the gene of interest and stratified into two groups where the uppermost quartile (>75%) were designated high expressers, and the lowermost quartile (<25%) were designated low expressers. Overall survival between these groups was analysed and plotted as Kaplan-Meier curves with p values determined using a log-rank test. Correlation analyses were then performed between the 2 genes of interests and the relationship expressed as r.

### Statistical Analysis

Statistical analysis was performed using Prism 7 (GraphPad Software). p values were determined and shown as *, **, ***, and ****, which represented p<0.05, 0.01, 0.001 and 0.0001 respectively.

### Fluorescence-Activated Cell Sorting (FACS™)

Conventional (Tconv) and regulatory (Treg) T cells were sorted by FACS from the spleen and liver of female Foxp3-RFP^{+/+} mice. CD4⁺ T cells were isolated by MACS using the CD4⁺ T cell isolation kit, mouse (Miltenyi Biotec) according to manufacturer's instructions. The flow through, containing enriched CD4⁺ T cells were stained with monoclonal anti-mouse fluorescein isothiocyanate (FITC)-conjugated TCRβ (H57-597) and allophycocyanin (APC)-conjugated CD4 (GK1.5) (both from BioLegend). Tconv cells were identified as TCRβ⁺ CD4+ RFP⁻ while Treg cells were identified as TCRβ⁺ CD4⁺ RFP⁺. After sorting, cells were stored in buffer RLT (QIAGEN) at −80° C.

### Real-Time Quantitative Polymerase Chain Reaction (RT-qPCR)

Foxp3⁺ and Foxp3⁻ cells sorted from naïve or infected Foxp3-RFP^{+/+} mice were stored in buffer RLT and homogenized in QIAshredder columns (both from QIAGEN®). RNA was extracted from using the RNeasy Mini Kit (QIAGEN®) according to manufacturer's instructions. The concentration of RNA (ng/μ1) and sample purity (260/280 ratio) was measured using the Nanodrop 2000 UV-Vis Spectrophotometer (Thermo Fisher Scientific). Extracted RNA was reverse transcribed to complementary DNA (cDNA) using the High-Capacity cDNA Reverse Transcription Kit (Applied Biosystems™) as per manufacturer's instructions. QuantiTect Primer Assays (specific for M. musculus B2m, Hprt, Nkg7, and Map3k8; QIAGEN®) or RT² qPCR Primer Assays (specific for M. musculus Cdkn1a; QIAGEN®) were used with the GoTaq qPCR Master Mix (Promega Corporation) in a final reaction volume of 10 μl containing 1 μl of template cDNA. RT-qPCR was performed in Hard-Shell® 384-Well Plates, thin wall, skirted, clear/clear (Bio-Rad, Hercules Calif., U.S.A.), sealed with Microseal® ‘B’ PCR Plate Sealing Film, adhesive, optical (Bio-Rad) on the QuantStudio 5 Real-Time PCR System (Applied Biosystems). Relative quantification was performed using the comparative C_{T} method relative to the average of two internal control genes: B2m and Hprt.

For human samples, CD4+ T cells were enriched by MACS using the CD4 MicroBeads, human (Miltenyi Biotec) according to manufacturer's instructions. RNA was then extracted, and reverse transcribed to cDNA. Relative quantification was performed using the comparative C_{T} method relative to 18S ribosomal RNA (rRNA).

### In Vitro Immune Stimulation Assay

Splenic mononuclear cells were isolated from mice, as described in the section: ‘Preparation of spleen single cell suspensions’. 4×10⁵ cells were cultured in each well of a 96-well U-bottom plate either in the presence of 4 μg/ml purified anti-mouse (α)-CD3 (pre-coated onto wells for 2 hours at 37° C.)+2 μg/ml α-CD28+20 ng/ml recombinant mouse (r)IL-2 (all from BioLegend), 1 μg/ml lipopolysaccharides from E. coli 0111:B4 (LPS) (Sigma-Aldrich), or 15 μg/ml ODN 1826 (CpG) (Sigma-Aldrich) for 24 hours at 37° C. with 5% CO₂. All stimulants were prepared in T cell media made up of Dulbecco's Modified Eagle Medium (DMEM; 4.5 g/L D-Glucose, L-Glutamine, 110 mg/L (1 mM) Sodium Pyruvate; Gibco) with 10% fetal bovine serum (FBS; Gibco), 0.05 mM β-mercaptoethanol (Sigma-Aldrich), 1×MEM Non-Essential Amino Acids Solution (NEAA; Gibco), and 100 U/ml Penicillin and 100 μg/ml Streptomycin (Gibco). T cell media was used as the control condition. At 24 hours of culture, 50 μl of supernatant was collected from each well and stored at −20° C. until further use. Cytokine levels were measured using the Mouse Inflammation BD™ Cytometric Bead Array (CBA) kit (Becton Dickinson).

### Metastasis Models

Mice were injected intravenously (i.v.) with either 1×10⁵ B16F10 cells or 5×10⁵ LWT1 cells. Lungs were harvested from mice injected with B16F10 cells at day 14 post-injection. Lungs were harvested from mice injected with LWT1 cells at day 14 post-injection and perfused with India ink. Metastatic burden in both models were quantified by counting colonies on the lung surface under a light microscope.

### NK Cell Transfer

Groups of Rag2γc-KO mice were injected intravenously (i.v.) with 2×10⁵ C57BL/6N (B6N; wild-type) or B6N.Nkg7-KO NK cells isolated by fluorescence-activated cell sorting (FACS). Six days later, naïve or adoptively-transferred Rag2γc-KO mice received either 1×10⁵ or 1×10⁴ B16F10 cells. Lungs were harvested from mice injected with B16F10 cells at day 14 post-injection. Metastatic burden was quantified by counting colonies on the lung surface under a light microscope.

### Dextran Sodium Sulfate (DSS)-Induced Colitis

The starting weight of mice were recorded prior to administration of drinking water containing 2.5% DSS salt (MW ca 40,000; Alfa Aesar, Mass. U.S.A.). Mice were scored for symptoms of colitis and body weight was recorded at approximately the same time daily. Mice were scored and weighed twice daily when the % loss of body weight exceeded 10%. Drinking water containing DSS was removed on day 6 and mice were given fresh drinking water. All mice were sacrificed at day 7 post-administration of DSS, during which colons were measured and preserved in formalin for histology sections. Colons were then transferred to 70% ethanol and embedded in paraffin prior to sectioning. Haemotoxylin and Eosin (H&E) stained-sections were scored for parameters including crypt architecture, crypt abscesses, tissue damage, inflammatory cell infiltration, and the amount of laminar propria neutrophils.

Plasmodium berghei ANKA Infection and Bioluminesce Imaging

Luciferase-expressing P. berghei ANKA parasites were injected intraperitoneally (i.p.) into a C57BL/6J (wild-type) passage mouse. After 4 days p.i. when parasitemia was between 1-3%, the passage mouse was euthanised and blood was collected via cardiac puncture, into 5 ml RPMI/PS+5 U/ml Heparin. The parasite inoculum was prepared by adding 5 ml of RPMI/PS and centrifuging at 338 g for 7 minutes at room temperature. The supernatant was discarded and the red blood cell (RBC) pellet resuspended in 1 ml of RPMI/PS for counting. RBCs were prepared in a 0.1% Trypan Blue solution (diluted from 0.4% in 1×PBS; MP Biomedicals Pty Ltd, Seven Hills NSW, Australia) for counting. A parasite inoculum containing 5×10⁵ pRBCs/ml was prepared and mice were injected i.v. with 200 μl (1×10⁵ parasites per mouse). Parasitemia was monitored by flow cytometry using Hoechst 33342 (Sigma-Aldrich) and Syto® 84 Orange Fluorescent Nucleic Acid Stain. Mice were scored daily for symptoms of experimental cerebral malaria (ECM) including ruffling of fur, hunching, the presence of a wobbly gait, limb paralysis, and convulsions. At the onset of neurological symptoms, mice were anaesthetised using IsoThesia® NXT (Henry Schein, Melville N.Y., U.S.A.) and 100 μg of D-luciferin (Caliper, Waltham Mass., U.S.A.) i.p., and imaged using the Xenogen In Vivo Imaging System (IVIS®; PerkinElmer, Waltham Mass., U.S.A.) set on bioluminescence imaging, for a duration of 1 minute. Mice were perfused with 1×PBS through the heart and brains were harvested for bioluminescence imaging. Imaging analysis was performed using the Living Image 4 software (PerkinElmer).

### Example 2. Results

### Differential Expression of NKG7 During Infection

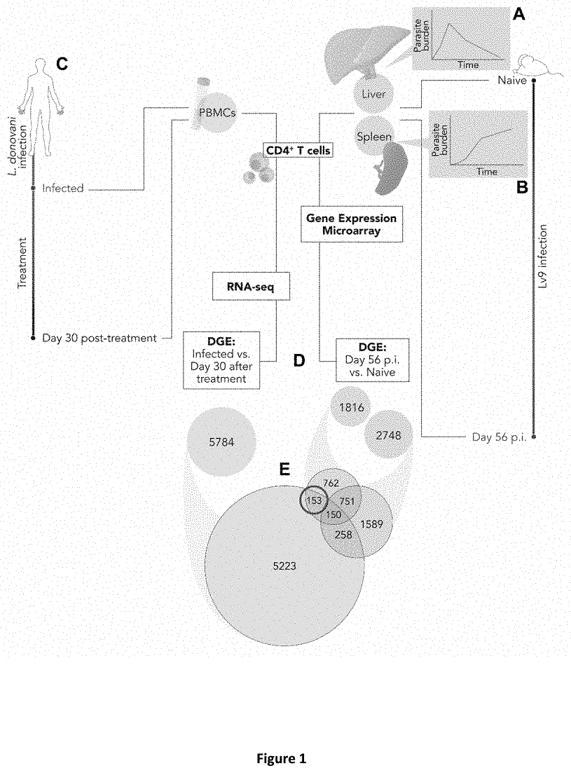

CD4⁺ T cells sorted from the liver and spleen of naïve and Leishmania donovani-infected C57BL/6 mice were isolated by fluorescence-activated cell sorting (FACS) (FIGS. 1A and 1B). The liver is a site of acute, resolving infection (immunity develops and the organ is protected against re-infection), while the spleen is a site of chronic infection, hyper-inflammation, and dysfunctional CD4⁺ T cells in C57BL/6 mice by day 56 post-infection. Gene expression by these tissue-specific CD4⁺ T cells was determined by whole genome microarray. Concurrently, CD4⁺ T cells from visceral leishmaniasis (VL) patients infected with L. donovani on presentation to clinic and 30 days after drug treatment were isolated and used to identify differentially expressed genes (DEGs) associated with human VL using RNAseq. A comparison was then made of infected mouse and human data sets with either corresponding naïve cell populations (mouse) or corresponding cell populations 30 days after drug treatment (patients) and found 1816, 2748, and 5784 DEGs in CD4⁺ T cells from the spleen, liver, and PBMCs (FIGS. 1D and 1E).

The DEGs from each source were compared to determine a core gene signature for CD4⁺ T cells during VL that was common between three sources and across two mammalian host species. This gene signature consisted of 150 genes. Based on the tissue-specific response to infection, a chronic signature of CD4⁺ T cells was identified as genes that were similarly upregulated or downregulated in the spleen and human PBMCs (FIG. 1, circled), whereas a resolving signature of CD4⁺ T cells was identified as the overlapping genes in the liver and human PBMCs. As seen in the Venn diagram in FIG. 1E, 153 and 258 differentially-expressed genes represented the chronic and resolving signature of CD4⁺ T cells during L. donovani infection, respectively. NKG7 was identified as the top-ranking, upregulated gene from the chronic signature.

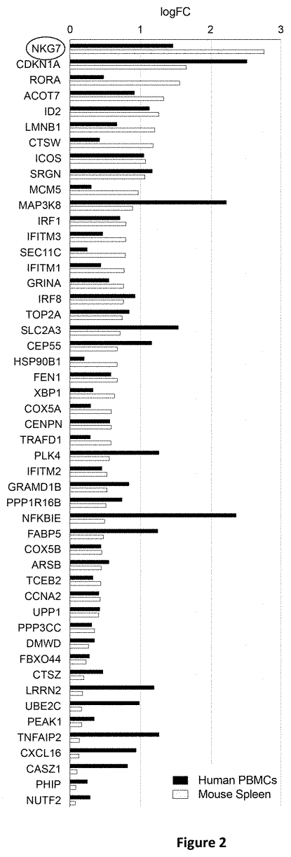

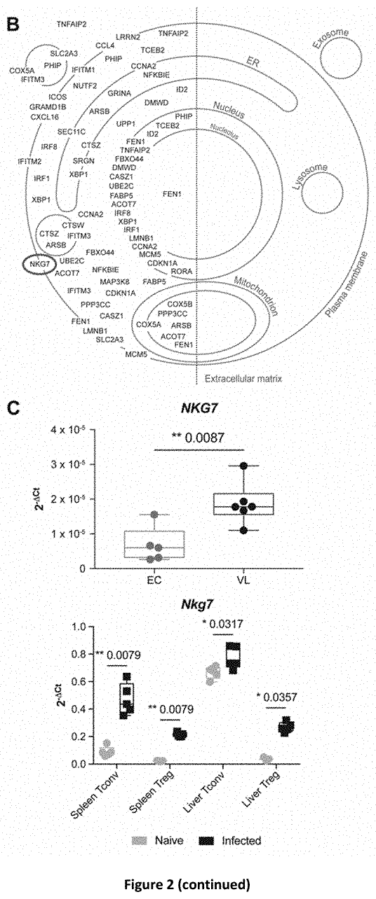

The chronic signature comprised 49 upregulated genes (FIG. 2A) with NKG7 being the most upregulated gene in CD4⁺ T cells isolated from mouse spleen (circled) and strongly associated with inflammation in this tissue. Cellular localisation of these molecules was determined by extracting information from the Gene Ontology (GO): Cellular Compartment (CC) terms (FIG. 2B). NKG7 is known to encode a membrane protein based on the GO:CC term “integral component of the plasma membrane”. Additionally, the GO term “protein binding” was associated with NKG7. The increase in NKG7 expression was validated by real-time qPCR in CD4⁺ T cells isolated from the PBMCs of patients with visceral leishmaniasis (VL) and endemic controls (EC), as well as Foxp3⁻ CD4⁺ T conventional (Tconv) cells and Foxp3⁺ CD4⁺ T regulatory cells (Tregs) from the spleen of L. donovani-infected mice (FIG. 2C). In line with the differential gene expression analysis data, NKG7 was significantly upregulated in VL samples when compared to EC samples (p=0.0087). Additionally, Nkg7 was significantly upregulated in the Tconv and Treg cells in the spleen and liver during infection. However, transcript expression of Nkg7 in naïve Tconv cells from the liver was higher than naïve splenic Tconv cells.

### NKG7 Expression in Cancer

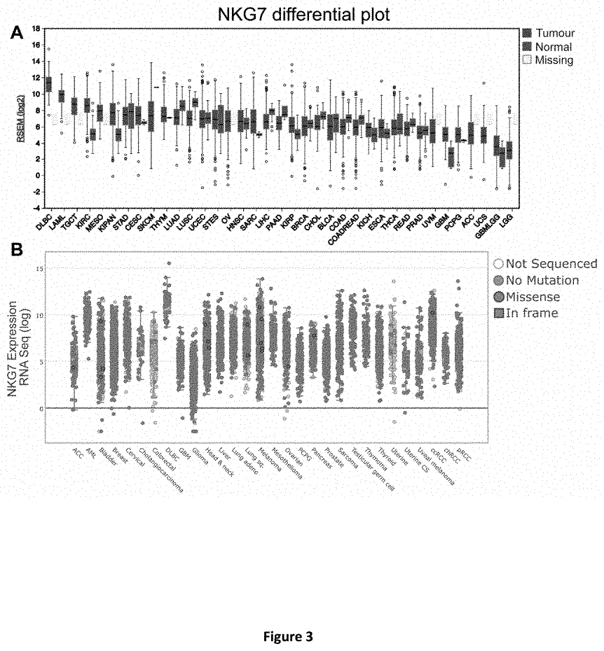

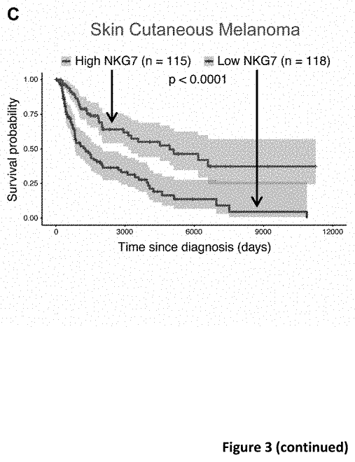

To investigate the potential role of NKG7 in cancer, NKG7 gene expression levels from 37 cancer datasets that are part of The Cancer Genome Atlas (TCGA) were obtained. Differential expression of NKG7 within the tumour was found, most notably in kidney renal clear cell carcinoma (KIRC) and Glioblastoma (GBM) (FIG. 3A; adapted from http://firebrowse.org/). While expression data was obtained from tumour biopsies, there was little evidence of mutation sites on NKG7 that confer cancer risk (FIG. 3B; adapted from http://www.cbioportal.org/output). Moreover, gene expression data to date suggests that NKG7 expression was by far the highest in the bone marrow and immune system compared to other tissues (https://www.proteinatlas.org/ENSG00000105374-NKG7/tissue).

To determine if differences in NKG7 expression was beneficial or detrimental, 470 patient samples from the skin cutaneous melanoma cohort (TGCA: SKCM) based on their expression of NKG7 were ordered. Stratification of patients into high NKG7 expression (top quartile, “High NKG7”) and low NKG7 expression (lowest quartile, “Low NKG7”) revealed a significant increase in survival probability in the group with high NKG7 expression (FIG. 3C (left); p<0.0001, shaded area indicates confidence interval). This indicates a broader clinical implication for targeting NKG7. Correlation analysis between NKG7 expression and CD4, CD8, or NCAM expression in the SKCM dataset was then performed to determine if NKG7 was associated with CD4-, CD8-, or NCAM-expression. These results showed that NKG7 expression was highly correlated with CD4 and CD8 but not NCAM expression.

### NKG7 Expression in B6.Nkg7-Cre Mouse

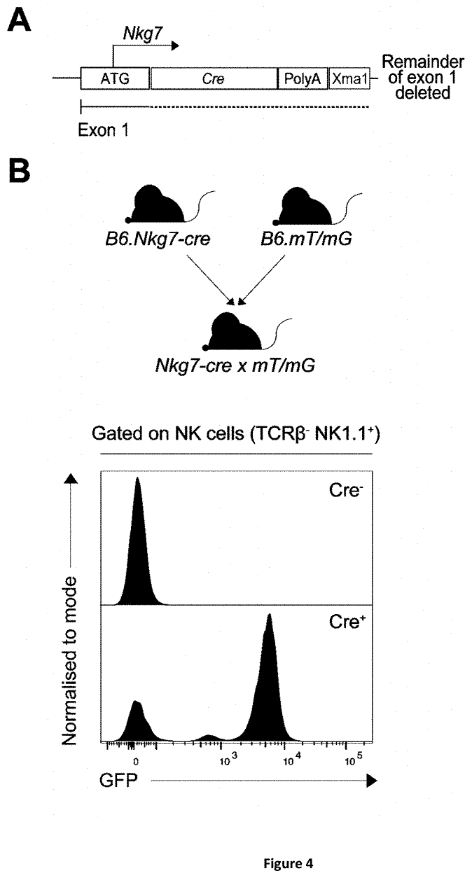

A B6.Nkg7-cre mouse was generated to characterise the expression or loss of Nkg7 in various contexts (FIG. 4A). To determine the expression of Nkg7, B6.Nkg7-cre mice were crossed with B6.membrane tdTomato/membrane green fluorescent protein (GFP) (mT/mG) mice to produce the Nkg7 reporter mouse Nkg7-cre×mT/mG (FIG. 4B). The expression of Nkg7 in Cre-expressing (+) mice, was determined by the surrogate marker GFP, and was validated on natural killer cells in the naïve state. t-Distributed Stochastic Neighbour Embedding (t-SNE) was performed to evaluate the expression of Nkg7 by key immune subsets in the naïve state. t-SNE plots demonstrated that the expression of GFP in the naïve state was limited to NK cells (NK1.1⁺ TCRβ⁻) and CD8⁺ T cells (NK1.1⁻ TCRβ⁺ CD4⁻ CD8⁺) (data not shown).

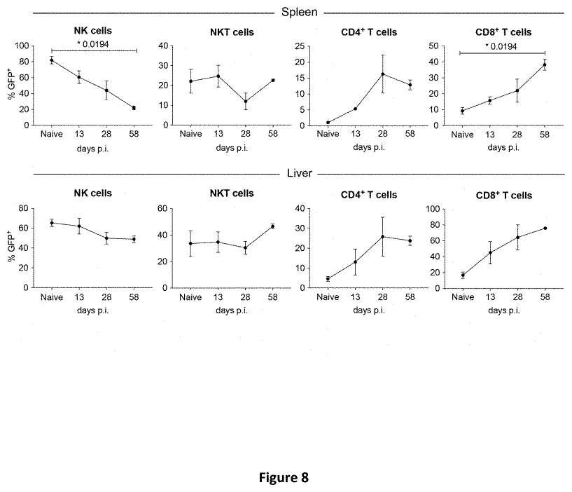

Nkg7-cre×mT/mG mice were infected with L. donovani and changes in the expression of GFP within each immune cell subset was tracked at days 13, 28, and 58 post-infection (p.i.). GFP expression within the NK cell population decreased as during the course of infection while the proportion of GFP CD4⁺ and CD8⁺ T cells increased (FIG. 8). These observations were consistent in both the spleen and liver. Statistical significance was determined using the Kruskal-Wallis test with Dunn's multiple comparisons test. * indicates p value and error bars represent mean±standard error of mean (SEM).

L. donovani Infection of B6.Nkg7-Ko Mice

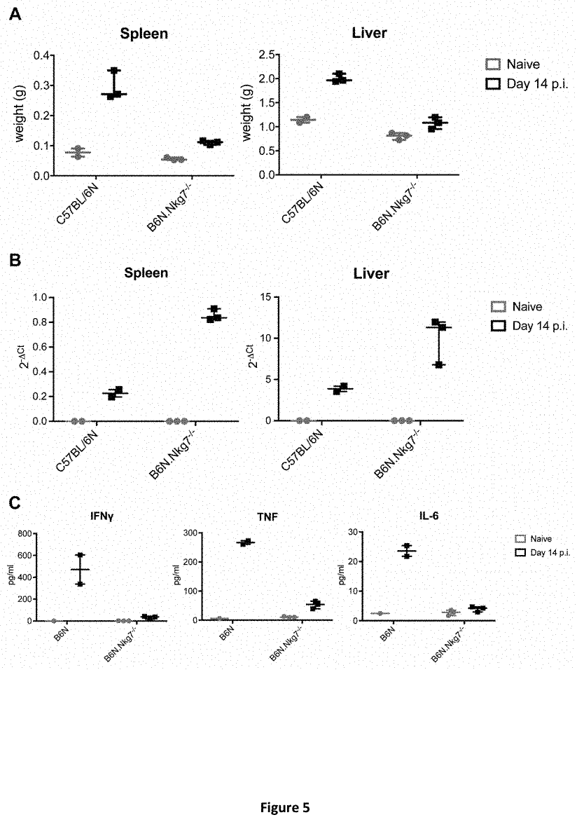

B6N.Nkg7-KO mice were infected with L. donovani and the effects of Nkg7 deficiency on host protection and pathology during visceral leishmaniasis were measured. The effects of Nkg7 deficiency at day 14 post infection (p.i.) were quantified and it was found that mice deficient in Nkg7 exhibited lower spleen and liver weights compared to control (wild-type) mice (FIG. 5A). This is indicative of lower inflammation levels resulting in less tissue damage. Parasite burden in Nkg7-deficient mice were markedly elevated compared to wild-type mice (FIG. 5B), suggesting that Nkg7 deficiency resulted in an impaired inflammatory response, and thus, a compromised ability of the host immune response to clear parasites. In agreement with these observations, levels of the pro-inflammatory cytokines interferon (IFN)γ, tumour necrosis factor (TNF), and IL-6 were found to be lower in the serum of Nkg7-deficient mice compared to control mice (FIG. 5C).

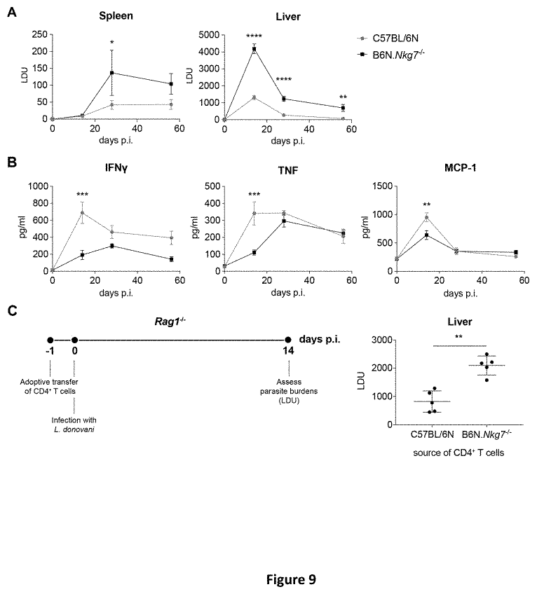

The parasite burden in the spleen and liver was measured at days 14, 28, and 58 p.i. (parasite burden expressed as Leishman-Donovan Units (LDU)) and increased liver parasite burdens at days 28, and 58 p.i., in addition to day 14 p.i. (FIG. 9A) were found. However, as was previously observed, the increase in the pro-inflammatory cytokines IFNγ, TNF, and MCP-1 was restricted to day 14 p.i. (FIG. 9B). Next, the inventors sought to identify the immune cell population that contributed to the observed phenotype. Given the major role of CD4⁺ T cells in the host immune response during L. donovani infection, CD4⁺ T cells were transferred either from C57BL/6N controls or B6N.Nkg7-KO donors into Rag1-KO mice. The increased parasite burden in the liver at day 14 p.i. was observed even when Nkg7-deficiency was restricted to CD4⁺ T cells, suggesting their contribution to the exacerbation of parasite burden in the absence of Nkg7 (FIG. 9C).

### Transmembrane Prediction of NKG7

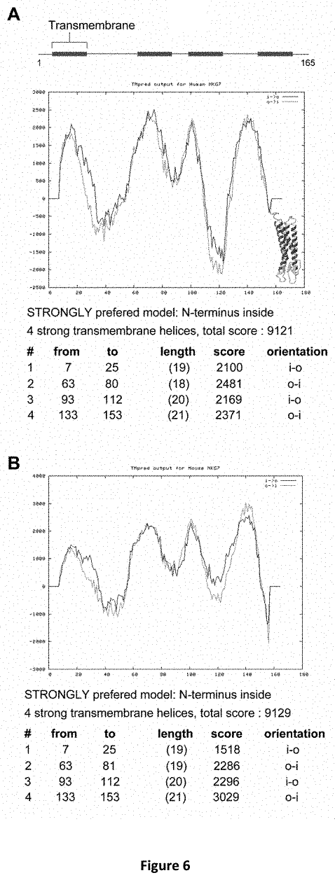

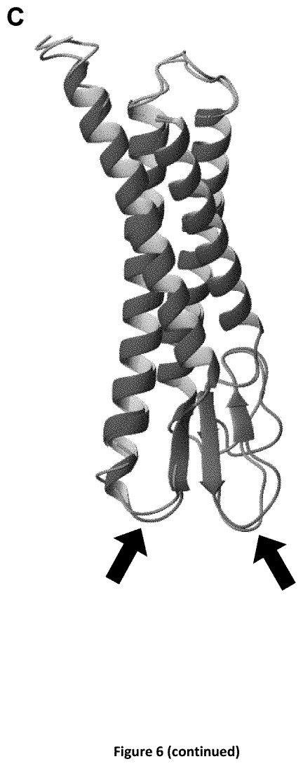

NKG7 was confirmed as being a transmembrane protein using the transmembrane prediction program TMpred. Predictions from TMpred for the human (FIG. 6A) and mouse (FIG. 6B) protein indicated the presence of four transmembrane helices and two extracellular loops that were likely to be expressed on the extracellular side. The predicted model of the NKG7 protein was consistent with the prediction by TMpred and showed two extracellular loops (indicated by the arrows) that were conserved in both human and mouse NKG7 (FIG. 6C). The amino acid sequence of the human NKG7 extracellular loops are provided in SEQ ID Nos:11 and 12, and the amino acid sequence of mouse NKG7 extracellular loops are provided in SEQ ID Nos:13 and 14.

### Nkg7-Deficiency Results in Elevated Levels of the Anti-Inflammatory Cytokine Interleukin (IL)-10 In Vitro

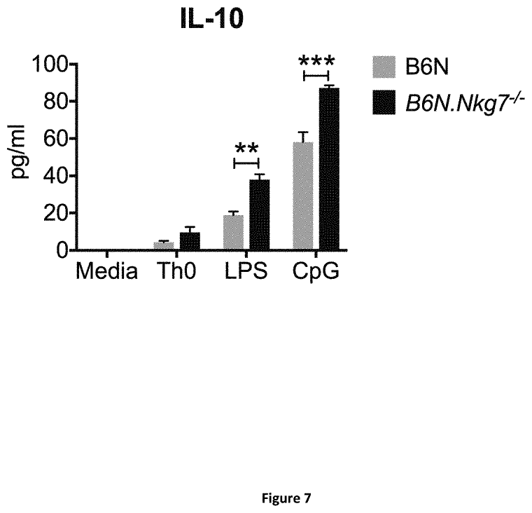

To determine the effects of Nkg7-deficiency in the context of immune stimulants, the present inventors isolated splenic mononuclear cells from C57BL/6N (B6N; wild-type) and B6N.Nkg7^{-/-}-KO mice and performed an in vitro stimulation with either purified monoclonal anti-mouse (α)-CD3+α-CD28+ recombinant (r)IL-2 (Th0 condition), lipopolysaccharide (LPS), or ODN 1826 (CpG) for 24 hours. Stimulation with the microbe-associated molecular patterns (MAMPs): LPS and CpG resulted in elevated IL-10 levels in the supernatants collected from B6N.Nkg7^{-/-} wells compared to B6N wells (FIG. 7).

### Nkg7-Deficiency Promotes Increased Metastases

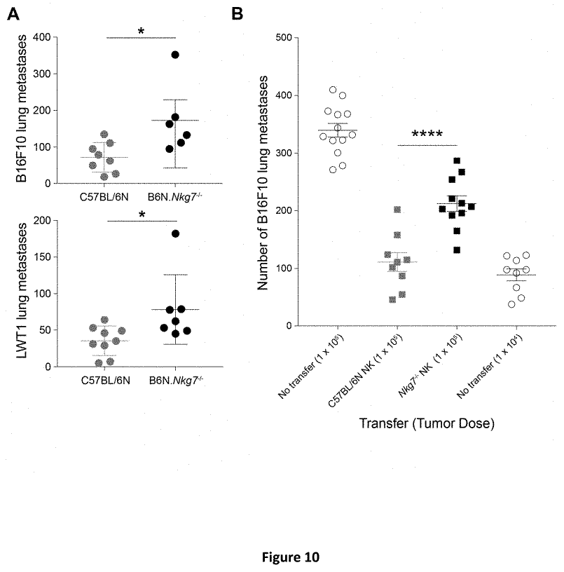

To investigate the role of Nkg7 in the context of cancer, either B16F10 or LWT1 cells were transferred into C57BL/6N (B6N; wild-type) and B6N.Nkg7-KO mice. Nkg7-deficiency resulted in increased lung metastases in both models (FIG. 10A). Given the importance of NK cells in the control of metastasis within these models, NK cells were isolated from B6N control mice or B6N.Nkg7-KO mice and transferred into Rag2γc-KO mice. The same phenoptype was observed even when Nkg7-deficiency was restricted to NK cells suggesting their role in increasing metastasis, in the absence of Nkg7 (FIG. 10B).Nkg7-deficiency reduces inflammation during dextran sodium sulfate (DSS)-induced colitis

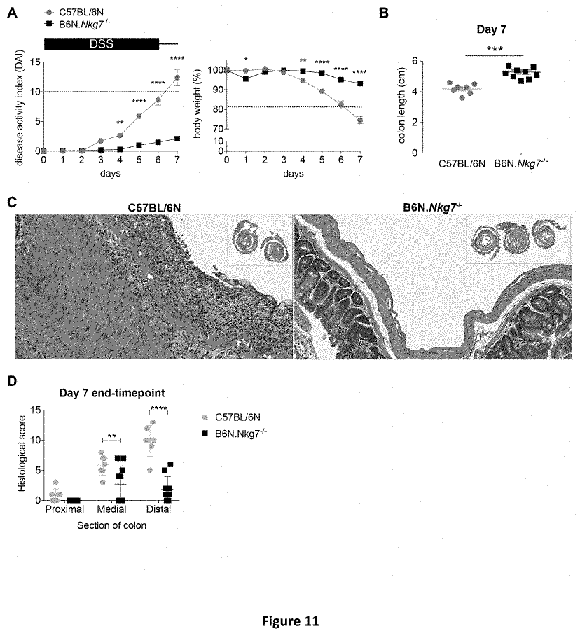

C57BL/6N (B6N; wild-type) and B6N.Nkg7-KO mice were given drinking water containing DSS for 6 days to induce colitis. The severity of symptoms (reported as the disease activity index (DAI)) and change in body weight were measured daily. B6N.Nkg7-KO mice exhibited less severe disease and a decreased loss of body weight when compared to wild-type mice (FIG. 11A). Shortening of the colon indicates inflammation and measurement of colon length at day 7 post-administration of DSS indicated less inflammation in B6N.Nkg7-KO mice relative to wild-type mice (FIG. 11B). Histological analysis of colon sections also indicate less inflammation in B6N.Nkg7-KO mice with intestinal crypts observed to be largely intact (FIG. 11C) and sections recording a lower histological score (FIG. 11D).

P. berghei ANKA Infection of of B6.Nkg7-Ko Mice

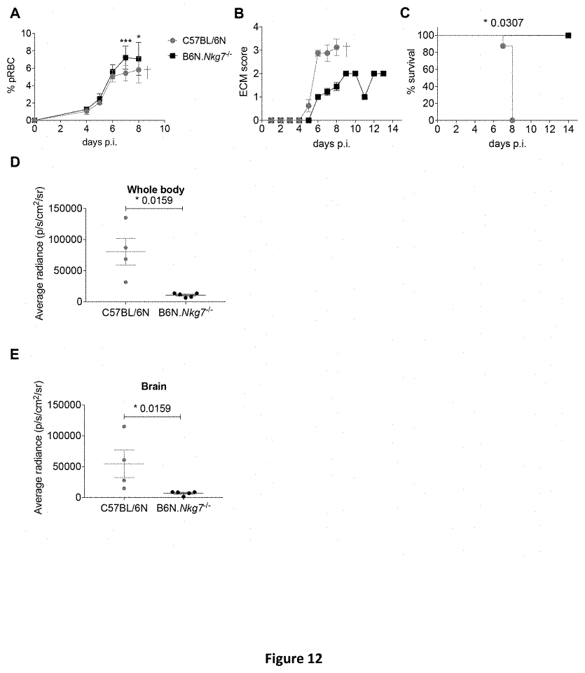

P. berghei ANKA infection is lethal in C57BL/6N (B6N; wild-type) mice due to the development of experimental cerebral malaria (ECM), which is mediated by inflammation. C57BL/6N (B6N; wild-type) and B6N.Nkg7-KO mice were infected with P. berghei ANKA to determine if Nkg7-deficiency had an effect on ECM. Although B6N.Nkg7-KO mice were found to have increased circulating parasitised red blood cells (pRBCs) (FIG. 12A), these mice exhibited less severe ECM symptoms (reported as ECM score; FIG. 12B) compared to wild-type mice. B6N.Nkg7-KO mice also survived up to day 14 p.i. when they suffered from symptoms of anaemia, but not ECM (FIG. 12C). Conversely, wild-type mice succumbed to ECM between days 7 and 8 p.i. The use of luciferase-expressing P. berghei ANKA parasites allowed for the measurement of whole body and brain parasite burden using bioluminescence imaging. B6N.Nkg7-KO mice showed decreased whole body (FIG. 12D) and brain (FIG. 12E) parasite burden relative to wild-type mice.

### SUMMARY

An RNA-seq dataset identified NKG7 to be one of the most differentially expressed gene in CD4⁺ T cells isolated from human peripheral blood mononuclear cells (PBMCs) and mouse spleen during Leishmania donovani infection. The present disclosure describes the generation of the tools needed to further characterise NKG7 as an immune target, including a mouse expressing CRE under the Nkg7 promoter. This B6.Nkg7-cre mouse, crossed to a membrane reporter allowed identification of immune cells that express Nkg7 both in vitro and in vivo at the steady state and in the context of L. donovani infection and graft-versus host disease (GVHD).

Furthermore, an inducer and a suppressor of NKG7 expression in CD4⁺ T cells have been identified. Using the Nkg7-KO mouse obtained from the KOMP repository, it was demonstrated that the loss of Nkg7 results in significantly reduced concentrations of pro-inflammatory cytokines in the blood during L. donovani infection.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

All publications discussed and/or referenced herein are incorporated herein in their entirety.

The present application claims priority to AU 2018901677, the entire contents of which are incorporated herein in their entirety.

Any discussion of documents, acts, materials, devices, articles, or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Andrews (2014) FastQC A Quality Control tool for High Throughput Sequencing Data
Anders et al. (2015) Bioinformatics, 31(2):166-169
Ashurst (2017) tSNE plots v1.3 DOI:https://zenodo.org/badge/DOI/10.5281/zenodo.1136332.svg
Colaprico et al. (2016) Nucleic Acids Res, 44(8):e71-e71
Dobin et al. (2013) Bioinformatics, 29(1):15-21
Dominguez (2016) Nature Reviews Molecular Cell Biology, 17(1):5-15
Du et al. (2008) Bioinformatics, 24(13):1547-1548
Hultquist et al. (2016) Cell Rep, 17(5):1438-1452
Khare et al. (2016) Nature, 537(7619):229-233
Krzywinski et al. (2009) Genome Res, 19(9):1639-45
Ledford (2015) Nature, 522 (7554): 20-4
Ritchie et al. (2013) Nucleic Acids Research, 43(7):e47-e47
Robinson et al. (2010) Bioinformatics, 26(1):139-140
Schmittgen et al. (2008) Nat Protoc, 3(6):1101-1108
Szklarczyk et al. (2015) Nucleic Acids Res, 43:447-452
Trapnell et al. (2010) Nature Biotechnology, 28:511
Trapnell et al. (2012) Nature Protocols, 7(3):562-578
Turman et al. (1993) Hum Immun, 36:24-40
Wagner et al. (2016) Nucleic Acids Research, 44(D1):D1036-44
Zhang et al. (2014) Human Molecular Genetics, (R1): R40-6

1. A method of modulating an immune response in a subject, the method comprising administering to the subject a compound that modulates NKG7 activity.

2. The method of claim 1, wherein the compound inhibits NKG7 activity.

3. The method of claim 2, wherein the compound is an antibody that binds NKG7 and inhibits NKG7 activity.

4. The method of claim 1, wherein the compound increases NKG7 activity.

5. The method of claim 4, wherein the compound is an antibody that binds NKG7 and increases NKG7 activity.

6. The method of claim 1, wherein the antibody binds extracellular loop 1 or extracellular loop 2 of NKG7.

7. A method of treating or preventing an autoimmune disease or inflammatory condition in a subject, the method comprising administering to the subject a compound that inhibits NKG7 activity.

8. The method of claim 7, wherein the autoimmune disease or inflammatory condition is selected from graft versus host disease (GVHD), rheumatoid arthritis, inflammatory bowel disease (IBD), lupus, neuroinflammation, multiple sclerosis, Parkinson's disease, Alzheimer's disease, and systemic inflammatory response syndrome (SIRS).

9. A method of reducing the risk of graft versus host disease (GVHD) in a transplant recipient receiving an allogeneic transplant, the method comprising administering a compound that inhibits NKG7 activity to the transplant recipient.

10. The method of claim 9, wherein the compound that inhibits NKG7 activity is administered to the transplant recipient prior to receiving the allogeneic transplant.

11. The method of claim 9, wherein the compound that inhibits NKG7 activity is administered to the transplant recipient at the time of, or after receiving the allogeneic transplant.

12. The method of claim 7, wherein the compound is an antibody that binds NKG7 and inhibits NKG7 activity.

13. A method of treating cancer in a subject, the method comprising administering to the subject a compound that increases NKG7 activity in the subject.

14. The method of claim 9, wherein the compound is an antibody that binds NKG7 and increases NKG7 activity.

15. The method of claim 13, wherein the subject is treated with a cancer immunotherapy.

16. The method of claim 15, wherein the cancer immunotherapy is selected from immune checkpoint inhibitor therapy and antibody therapy.

17. A method of enhancing an immune response to a vaccine antigen in a subject, the method comprising administering to the subject:
i) a compound that increases NKG7 activity, and
ii) a vaccine antigen.

18. The method of claim 1, wherein NKG7 comprises an amino acid sequence at least 90% identical to any one of SEQ ID Nos:1, 3, 5, 7, or 9 and has NKG7 activity.

19. The method of claim 1, wherein NKG7 is encoded by a nucleic acid molecule which hybridises under stringent conditions with a nucleic acid comprising a sequence selected from any of SEQ ID Nos:2, 4, 6, 8, or 10.

20. The method of claim 1, wherein NKG7 consists of any of SEQ ID NOs:1, 3, 5, 7, or 9 and having substitution of, or deletion or addition of, one or several amino acids.

21-25. (canceled)


SEQUENCE LISTING (14 sequences)

SEQ ID NO: 1 - protein, 165 aa, Homo sapiens
MELCRSLALLGGSLGLMFCLIALSTDFWFEAVGPTHSAHSGLWPTGHGDIISGYIHVTQTFSIMAVLWALVSVSFLVLSCFPSLFPPGHGPLVSTTAAFAAAISMVVAMAVYTSERWDQPPHPQIQTFFSWSFYLGWVSAILLLCTGALSLGAHCGGPRPGYETL

SEQ ID NO: 2 - DNA, 498 bp, Homo sapiens
atggagctctgccggtccctggccctgctggggggctccctgggcctgatgttctgcctgattgctttgagcaccgatttctggtttgaggctgtgggtcccacccactcagctcactcgggcctctggccaacagggcatggggacatcatatcaggctacatccacgtgacgcagaccttcagcattatggctgttctgtgggccctggtgtccgtgagcttcctggtcctgtcctgcttcccctcactgttccccccaggccacggcccgcttgtctcaaccaccgcagcctttgctgcagccatctccatggtggtggccatggcggtgtacaccagcgagcggtgggaccagcctccacacccccagatccagaccttcttctcctggtccttctacctgggctgggtctcagctatcctcttgctctgtacaggtgccctgagcctgggtgctcactgtggcggtccccgtcctggctatgaaaccttgtga

SEQ ID NO: 3 - protein, 142 aa, Homo sapiens
MELCRSLALLGGSLGLMFCLIALSTDFWFEAVGPTHSAHSGLWPTGHGDIISGHGPLVSTTAAFAAGKDSGLDWGIASQRIPAEELSHLSCPCPQPSPWWWPWRCTPASGGTSLHTPRSRPSSPGPSTWAGSQLSSCSVQVP

SEQ ID NO: 4 - DNA, 429 bp, Homo sapiens
atggagctctgccggtccctggccctgctggggggctccctgggcctgatgttctgcctgattgctttgagcaccgatttctggtttgaggctgtgggtcccacccactcagctcactcgggcctctggccaacagggcatggggacatcatatcaggccacggcccgcttgtctcaaccaccgcagcctttgctgcaggtaaggactctggactggactggggcatcgcgagccagcgaattcctgccgaggagctgagccatctctcttgtccttgtccccagccatctccatggtggtggccatggcggtgtacaccagcgagcggtgggaccagcctccacacccccagatccagaccttcttctcctggtccttctacctgggctgggtctcagctatcctcttgctctgtacaggtgccctga

SEQ ID NO: 5 - protein, 85 aa, Homo sapiens
MELCRSLALLGGSLGLMFCLIALSTDFWFEAVGPTHSAHSGLWPTGHGDIISGHGPLVSTTAAFAAGALSLGAHCGGPRPGYETL

SEQ ID NO: 6 - DNA, 258 bp, Homo sapiens
atggagctctgccggtccctggccctgctggggggctccctgggcctgatgttctgcctgattgctttgagcaccgatttctggtttgaggctgtgggtcccacccactcagctcactcgggcctctggccaacagggcatggggacatcatatcaggccacggcccgcttgtctcaaccaccgcagcctttgctgcaggtgccctgagcctgggtgctcactgtggcggtccccgtcctggctatgaaaccttgtga

SEQ ID NO: 7 - protein, 165 aa, Mus musculus
MEPCRSLALFAGSLGLTSSLIALTTDFWIVATGPHFSAHSGLWPTSQETQVAGYIHVTQSFCILAVLWGLVSVSFLVLSCIPALSAPGRGPLVSTVMAFSAALSILVAMAVYTSMRWSQTPFSQVQTFFSWSFYLGWVSFILFLFAGCLSLGAHCRTRRAEYETL

SEQ ID NO: 8 - DNA, 498 bp, Mus musculus
atggagccctgccggtccctggccctgtttgctggctctctgggcctgacttcttctctgattgctctgaccactgatttctggatagtggccacaggtcctcacttctctgcccactctggcctctggccaacaagccaagagactcaagtagcaggttatatccatgtgacacagagcttctgtatcctggctgtcctgtggggcctggtgtctgtgagcttcctggttctgtcttgcatcccagccctgtctgctcccggccgtggccctctggtctcaactgtcatggctttttctgcagctctctccatattagtggctatggcagtgtacaccagtatgagatggagccagactccattttctcaagtccagacattcttctcctggtccttctacctaggctgggtctccttcatcctcttcctctttgcaggctgcctgagcctgggtgctcactgtagaacccgtcgggctgagtatgaaaccttgtga

SEQ ID NO: 9 - protein, 120 aa, Mus musculus
MEPCRSLALFAGSLGLTSSLIALTTDFWIVATGPHFSAHSGLWPTSQETQVAGYIHVTQSFCILAVLWGLVSVSFLVLSCIPALSAPGRGPLVSTVMAFSAGCLSLGAHCRTRRAEYETL

SEQ ID NO: 10 - DNA, 363 bp, Mus musculus
atggagccctgccggtccctggccctgtttgctggctctctgggcctgacttcttctctgattgctctgaccactgatttctggatagtggccacaggtcctcacttctctgcccactctggcctctggccaacaagccaagagactcaagtagcaggttatatccatgtgacacagagcttctgtatcctggctgtcctgtggggcctggtgtctgtgagcttcctggttctgtcttgcatcccagccctgtctgctcccggccgtggccctctggtctcaactgtcatggctttttctgcaggctgcctgagcctgggtgctcactgtagaacccgtcgggctgagtatgaaaccttgtga

SEQ ID NO: 11 - protein, 37 aa, Homo sapiens
DFWFEAVGPTHSAHSGLWPTGHGDIISGYIHVTQTFS

SEQ ID NO: 12 - protein, 20 aa, Homo sapiens
TSERWDQPPHPQIQTFFSWS

SEQ ID NO: 13 - protein, 37 aa, Mus musculus
DFWIVATGPHFSAHSGLWPTSQETQVAGYIHVTQSFC

SEQ ID NO: 14 - protein, 20 aa, Mus musculus
TSMRWSQTPFSQVQTFFSWS